(19) United States
(12) Patent Application Publication
(10) Pub. No.: US 2024/0382444 A1
(43) Pub. Date: Nov. 21, 2024

(54) COMPOSITIONS AND USE THEREOF FOR RE-ESTABLISHING INTESTINAL PERMEABILITY AND/OR PREVENTING OR COMBATTING MULTIFACTORIAL DISEASES

(71) Applicant: HYDRO FILL TECHNOLOGY, BOULIAC (FR)

(72) Inventor: Jean-Pascal ZAMBAUX, BOULIAC (FR)

(21) Appl. No.: 18/289,789

(22) PCT Filed: May 10, 2022
(86) PCT No.: PCT/EP2022/062638

§ 371 Date: Jul. 9, 2024

(30) Foreign Application Priority Data
May 10, 2021 (FR) FR 2104928

(51) Int. Cl. A61K 31/201; A61K 31/19; A61K 31/198; A61K 31/20; A61K 31/513; A61K 31/575; A61K 47/64; A61P 1/00

(52) U.S. Cl. CPC A61K 31/201; A61K 31/19; A61K 31/198; A61K 31/20; A61K 31/513; A61K 31/575; A61K 47/6455; A61P 1/00

(57) ABSTRACT

The present invention relates to a composition comprising at least the following molecules: oleic acid, palmitic acid, lauric acid, linoleic acid, azelaic acid, farnesylcysteine, palmitoleic acid, cholesterol, thioctic acid, myristic acid, orotic acid, acetic acid, butyric acid, lactic acid, propionic acid, and/or a salt and/or an ester and/or an anhydride of one or more of these molecules, and use thereof in particular as medicine.

### TECHNICAL FIELD

The invention relates to the prevention and/or treatment of multifactorial diseases. In particular, the invention relates to compositions comprising multiple selected specific molecules and to the use thereof in decreasing intestinal permeability and/or re-establishing the function of normal intestinal permeability and/or in preventing or combating various multifactorial diseases involving intestinal permeability.

### PRIOR ART

Most diseases, and in particular chronic diseases, are multifactorial. They always involve a genetic component and immunological and toxic components linked to “the environment”, i.e. bacteria, viruses, chemical agents, etc.

Among known multifactorial chronic diseases, particular mention can be made of degenerative diseases such as Parkinson's disease, Alzheimer's disease, post-stroke inflammation, amyotrophic lateral sclerosis, Huntington's disease, etc.

Degenerative diseases are diseases that case the progressive deterioration of one or more organs. Often of genetic origin, they can be triggered by high and prolonged exposure to biological and/or toxic products. Specifically, any bacterial constituents and/or other toxins crossing the mucous membranes excessively leads to activation of the immune system, which, in patients suffering from chronic conditions, always triggers the stimulation of autoreactive clones that attack target tissues (degeneration). Degenerative diseases are therefore intimately linked to intestinal hyperpermeability, like many infectious diseases and in particular viral diseases.

In a healthy human intestine, small particles (<4 angstroms in radius) can migrate through the pores in claudins which are the most important component of tight junctions, but particles ranging up to 10-15 angstroms (3.5 LDa) can pass via the paracellular absorption pathway. The intestine normally has a certain degree of permeability, which allows nutrients to cross the intestine while maintaining a barrier function to prevent potentially harmful substances from leaving the intestine and migrating further into the body.

The intestinal flora continuously interacts with the intestinal immune system, with which it is in contact. The interaction between the considerable body of antigens constituted by the intestinal microflora and the intestinal immune system is extremely complex, but in a balanced body, this maintains “immune background noise”, an essential balance between the bacteria of the flora and the immune system which regulates them.

An unbalanced flora, which is called dysbiosis, leads to an exacerbated response of the intestinal immune system, increasing inflammation, resulting in alteration of the tight junctions. In response to fragments of the walls of bacteria destroyed by the intestine, known as endotoxins, passing into general circulation, there is a substantial release of inflammatory cytokines by the body, and if there is a large influx of endotoxins, this can trigger the onset of septic shock, respiratory failure, renal ischemia or a cytokine storm. A blood endotoxin assay is also a reliable marker of intestinal hyperpermeability.

The causes of intestinal hyperpermeability are extremely numerous. Any disruption of the intestinal biotope, any dysbiosis, leads to a response from the immune system, with inflammatory cytokines being released, which results in the mucous membrane of the small intestine and of the colon being in an inflammatory state, with alteration of the epithelium. The causes of such dysbiosis can be, in particular:
stress, which greatly reduces the volume and quality of all digestive secretions,
emotional and psychological shock,
substantial strain or physical shock
antibiotic treatment,
viral, bacterial or parasitic infection,
radiation
toxic chemical agents: air pollution or water or food contamination; pesticides and heavy metals; drugs (statins, anti-inflammatoires, immunosuppressants, etc.).

Intestinal hyperpermeability, in addition to playing a role in triggering degenerative diseases in genetically predisposed patients, is a cause of inflammatory, infectious and autoimmune diseases, as well as allergies and severe food tolerances.

Currently, the proposed solutions for preventing or combating dysbiosis consist essentially in administering probiotics and/or prebiotics, but their efficacy is highly variable and depends on the patient's basic intestinal flora.

Moreover, with regard to degenerative diseases specifically, these are often very hard on the patient. Some symptoms may be reduced, but such diseases are never completely cured and there is currently no satisfactory treatment. Many chemical agents have therapeutic properties but their toxicity, their lifetime, or their rapid elimination make them unsuitable for treating degenerative diseases.

Specifically, despite them being multifactorial, current treatments target just one of the causal factors of these diseases, and do not seek to act on intestinal hyperpermeability. For example, amyotrophic lateral sclerosis is linked to numerous factors, in particular to oxidative stress, mitochondrial dysfunction, neuroinflammation, excitotoxicity, oligodendrocyte dysfunction and degeneration, altered proteostasis, an altered DNA repair system, changes in nucleocytoplasmic RNA and in RNA related to transport proteins, defective axonal transport, and defective vesicular transport. Currently, the treatment recommended for amyotrophic lateral sclerosis is Riluzole® which acts only on the inhibition of glutamate release in order to combat excitotoxicity, and no action on intestinal permeability is considered. The same observation can be made for other degenerative diseases.

There is therefore a need for compositions capable of acting on various factors in order to prevent and/or combat the effects of intestinal hyperpermeability and associated multifactorial diseases, in particular degenerative, autoimmune, inflammatory or infectious diseases.

### SUMMARY OF THE INVENTION

To address this need, the invention proposes a composition that simultaneously:
reduces the inflammatory process
decreases intestinal permeability
has an antioxidant effect
has an antiradical effect
has an antibacterial, antiviral and antifungal effect,
and preferentially rebalances the neurological metabolisms causing the degeneration of neurons and motor neurons.

To this end, the invention relates to a composition comprising at least the following molecules:
oleic acid,
palmitic acid,
lauric acid,
linoleic acid,
azelaic acid,
farnesyl cysteine,
palmitoleic acid,
cholesterol,
thioctic acid,
myristic acid,
orotic acid,
acetic acid,
butyric acid,
lactic acid,
propionic acid,
   and/or a salt and/or an ester and/or an anhydride of one or more of these molecules.

Advantageously, these molecules each act on different factors, allowing combined action on the inflammatory process, intestinal permeability, toxic chemical agents, free radicals and toxic microorganisms. The molecules act together to restore the function of intestinal permeability which has been disrupted by an imbalanced microbiota.

To increase the efficacy and bioavailability of these molecules, some are preferentially conjugated to specific polymers and others, which cannot be conjugated directly to these polymers, are preferentially in the form of copolymers or are encapsulated in micelles.

The invention also relates to a method for producing these compositions.

The compositions according to the invention can be used as a drug alone or in combination with the use of at least one other composition, and the invention also relates to the compositions for their use as a drug.

Other features and advantages will become apparent from the detailed description of the invention, the examples and the figures that follow.

### BRIEF DESCRIPTION OF THE FIGURES

FIG. 1a shows a polymerization scheme: L-Met NCA and L-Lys NCA (trifluoroacetate) are mixed and polymerized to obtain the copolymer poly-(L-lysine-co-methionine). (DMF=dimethylformamide).

FIG. 1b shows a deprotection scheme: the copolymer of FIG. 1a is deprotected to obtain a poly-(L-lysine-co-methionine) in the form of a bromide salt.

FIG. 2a shows a polymerization scheme: L-Cys NCA (trityl) and L-Lys NCA (trifluoroacetate) are mixed and polymerized to obtain the copolymer poly-(L-lysine-co-L-cysteine).

FIG. 2b shows a deprotection scheme: the copolymer of FIG. 1a is deprotected to obtain a poly-(L-lysine-co-L-cysteine) in the form of a bromide salt. (DMF=dimethylformamide).

FIG. 3 shows a diagram of micelle formation according to one embodiment of the invention: 10=micelles; 12=polymer (polylysine for example); 14=covalently conjugated molecules (in particular fatty acids for example); 16=highly hydrophobic molecules which cannot be conjugated to polylysine (for example cholesterol, farnesyl cysteine, coenzyme Q10).

FIG. 4 is a graph showing a PLS-DA comparing the microbiota of WT and SOD1 mice

FIG. 5 is a graph showing a comparative analysis of the beta diversity of the intestinal microbiota of mice via the MDS—Jaccard method according to treatment.

FIG. 6 is a graph showing a comparative analysis of the beta diversity of the intestinal microbiota of mice via the MDS—Bray-Curtis method according to treatment.

FIG. 7 is a graph showing a comparative analysis of the intestinal microbiota of mice according to treatment via PLS-DA.

FIG. 8 is a graph showing an analysis of correlation between WT or SOD1 mice according to bacterial family.

FIG. 9 is a graph showing an analysis of correlation between SOD1 mice treated or untreated with a low dose.

### DETAILED DESCRIPTION OF THE INVENTION

### Definition

For the purposes of the invention, the term “animal” means any animal apart from humans. It can be a mammal in particular.

For the purposes of the invention, the term “amphiphilic conjugate” means a conjugate formed by a hydrophobic molecule X and a hydrophilic polymer Y, the conjugate thus having an amphiphilic character.

For the purposes of the invention, the term “molecule X conjugated to a polymer Y” means a molecule X covalently bonded to a polymer Y, this bond preferentially being an amide, urea or carbamate bond depending on the chemical nature of the molecule X and of the polymer Y.

### Composition C1

The present invention therefore relates to a composition C1 comprising at least the following active molecules:
oleic acid,
palmitic acid,
lauric acid,
linoleic acid,
azelaic acid,
farnesyl cysteine,
palmitoleic acid,
cholesterol,
thioctic acid,
myristic acid,
orotic acid,
acetic acid,
butyric acid,
lactic acid,
propionic acid,
   and/or a salt and/or an ester and/or an anhydride of one or more of these molecules.

Composition C1 can optionally also comprise pyruvic acid and/or a salt and/or an ester and/or an anhydride.

For all of the active molecules present in composition C1, when they are mentioned in the present application, they can be the molecules (e.g. butyric acid) per se and/or a salt (example: butyrate) and/or an ester and/or an anhydride of these molecules (e.g. butyric acid ester) and/or an anhydride.

### Preferentially:

the amount of butyric acid in composition C1 is greater than that of each of the other molecules taken individually, and/or
the amount of thioctic acid in composition C1 is greater than that of each of the other molecules taken individually, except for butyric acid, and/or
the amount of lauric acid in composition C1 is greater than that of each of the following molecules taken individually: palmitic acid, linoleic acid, azelaic acid, farnesyl cysteine, palmitoleic acid, cholesterol, myristic acid, and orotic acid,
the amount of oleic acid on the one hand, and of lactic acid on the other hand, in composition C1 is greater than that of each of the following molecules taken individually: palmitic acid, lauric acid, linoleic acid, azelaic acid, farnesyl cysteine, palmitoleic acid, cholesterol, myristic acid, orotic acid, and acetic acid.

Preferentially, each of the active molecules in composition C1 represents between 0.5 E-05 M and 10 E-05 M.

According to one embodiment, in order to improve the solubility, efficacy and bioavailability of the molecules of composition C1, at least one of the molecules of the composition selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids is covalently conjugated to at least one molecule of a polymer selected from among polylysine, polyethylene glycol, polyornithine, polyarginine and polyhistidine.

Specifically, the molecules selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids can all be covalently conjugated to a polymer selected from among polylysine, polyethylene glycol, polyornithine, polyarginine and polyhistidine, in particular via an amide, urea or carbamate bond.

According to one particularly suitable variant, all of the molecules selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids and esters of these acids, present in composition C1 according to invention, are covalently conjugated to at least one molecule of a polymer selected from among polylysine, polyethylene glycol, polyornithine, polyarginine and polyhistidine.

Among all of the molecules to be conjugated, two of them are highly hydrophobic (logP values greater than 1, preferentially greater than 2 and preferentially greater than 3) and are chemically not suitable for covalent conjugation either due to the absence of reactive functional groups or due to chemical incompatibility. These are cholesterol and farnesyl cysteine. However, given both the hydrophilic character of certain polymers (such as polylysine, polyethylene glycol, polyornithine, polyarginine or polyhistidine) and the hydrophobic character of the majority of the molecules suitable for covalent conjugation (oleic acid, palmitic acid, lauric acid, linoleic acid, azelaic acid, palmitoleic acid, thioctic acid, myristic acid, orotic acid, acetic acid, butyric acid, lactic acid, and propionic acid), then, according to the invention, these particular conjugates behave as surfactants in aqueous solution. In this way, some of these (amphiphilic) conjugates act as polymeric micelles in an aqueous medium, creating a hydrophobic pocket capable of encapsulating hydrophobic species, thereby considerably improving their solubility and stability in aqueous solution.

Farnesyl cysteine and cholesterol, and esters, salts and anhydrides of farnesyl cysteine or cholesterol, cannot be covalently bonded, i.e. cannot be conjugated, to a polymer selected from among polylysine, polyethylene glycol, polyornithine, polyarginine and polyhistidine. Thus, preferentially, farnesyl cysteine and/or cholesterol, and/or esters thereof, present in composition C1 are encapsulated in micelles. Thus, composition C1 preferentially comprises micelles in which at least farnesyl cysteine and/or cholesterol and/or one of the esters and/or salts and/or anhydrides thereof are encapsulated. According to one particularly suitable embodiment, at least one of the micelles is formed by amphiphilic conjugates each consisting of at least one hydrophobic molecule covalently conjugated to a molecule of a polymer selected from among polylysine, polyethylene glycol, polyornithine, polyarginine and polyhistidine. Even more preferentially, as shown in FIG. 3, at least one of the micelles 10 is formed by amphiphilic conjugates each consisting of at least one hydrophobic molecule 14 preferentially selected from among the fatty acids of the composition and in particular at least one molecule selected from among oleic acid, palmitic acid, lauric acid, linoleic acid, palmitoleic acid, myristic acid, salts, esters and anhydrides of these acids covalently conjugated to a molecule of a polymer 12 selected from among polylysine, polyethylene glycol, polyornithine, polyarginine and polyhistidine, said micelles encapsulating farnesyl cysteine and/or cholesterol and/or esters and/or salts and/or anhydrides 16 thereof. Specifically, oleic acid, palmitic acid, lauric acid, linoleic acid, azelaic acid, palmitoleic acid, myristic acid, propionic acid, salts of these acids, esters of these acids and anhydrides of these acids are hydrophobic and polylysine, polyethylene glycol, polyornithine, polyarginine and polyhistidine are hydrophilic polymers.

Thus, composition C1 preferentially comprises:
conjugates of oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids with polylysine, polyethylene glycol, polyornithine, polyarginine or polyhistidine, and
micelles formed by at least one of these conjugates, said micelles encapsulating farnesyl cysteine and cholesterol.

In particular, this configuration makes it possible:
to increase the half-life of the active molecules of the composition in the body,
to target tissues or cells on which the molecules of the composition are to act,
to allow the active molecules of the composition to cross the blood-brain barrier and to enter cells, in particular neurons and motor neurons,
to increase the stability and bioavailability of the active ingredient.

The efficacy of the composition is therefore enhanced and it is possible to administer lower doses and reduce the acute or chronic toxicity of the active molecules contained in the composition.

Preferentially, the one or more polymers conjugated to the molecules are selected from among poly-L-lysine, polyethylene glycol, poly-L-ornithine, poly-L-arginine and poly-L-histidine.

According to one embodiment, it is polylysine, preferentially poly-L-lysine. The polylysine is preferentially linear. In particular, the polylysine used is an epsilon poly-L-lysine with a molecular weight of between 12000 and 20000 Da.

Thus, according to one variant, composition C1 comprises at least:
A. the following conjugates, each conjugate consisting of a molecule covalently bonded to a polylysine:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates
one or more linoleyl-poly-L-lysine conjugates, and
B. farnesyl cysteine and cholesterol, and/or an ester of these molecules, encapsulated in micelles, preferentially in micelles formed by one or more of the conjugates listed in list A.

In this composition, the poly-L-lysine can be replaced with another polylysine or with polyethylene glycol, a poly-L-ornithine, a poly-L-arginine or a poly-L-histidine.

Composition C1 according to the invention can be in solid form or in liquid form. When in liquid form, the composition comprises at least water and the constituents mentioned above. The solid form is preferentially obtained from the liquid form, preferentially by freeze-drying. Thus, when composition C1 in liquid form comprises micelles and it is freeze-dried into solid form, the micelles reform when composition C1 in solid form is placed back into an aqueous solution.

Composition C1 also preferentially comprises pharmaceutically acceptable excipients. These excipients can be selected in particular to meet the pH and osmolarity requirements of solutions for injection into humans or animals. For example, they can be acids or bases for adjusting pH or NaCl for adjusting osmolarity.

Composition C1 according to the invention is intended to be administered to human beings, or to an animal, and is consequently in a form suitable for such administration. When it is in liquid form, it is preferentially suitable for subcutaneous or intravenous administration, in particular intravenous infusion, and it is packaged in suitable containers known to a person skilled in the art for packaging this type of product. Composition C1 according to the invention can also be administered in liquid form via a pump, like an insulin pump.

When it is in solid form, it is preferentially suitable for administration:
transcutaneously and it is preferentially formulated and packaged in the form of a patch, or
nasally in the form of a powder, or
sublingually in the form of a powder or tablet, or
transmucosally, and it is preferentially formulated and packaged in the form of a mucoadhesive tablet.

### Method for Producing Composition C1

Composition C1 according to the invention can be produced using any suitable method.

If the molecules that constitute composition C1 are used as they are in a solvent, they can all be mixed together in said solvent.

If the molecules in composition C1 are conjugated to polymers for some and in micelles for others, the production method can comprise the following steps:
a. creating an amphiphilic premix: mixing, in an aqueous solution, one or more conjugates selected from among:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates,
one or more linoleyl-poly-L-lysine conjugates, and
b. adding at least farnesyl cysteine and cholesterol to the amphiphilic premix and stirring so as to form micelles formed by one or more of the conjugates of the amphiphilic premix encapsulating the farnesyl cysteine and cholesterol.

Specifically, one variant of the invention consists in taking advantage of the amphiphilic nature of the majority of the individual components to create an amphiphilic premix which allows the controlled solubilization of the hydrophobic species. The solubilization process according to one preferred embodiment consists in the controlled addition of the hydrophobic molecules to the amphiphilic premix and allowing the time needed for their dissolution with stirring.

The poly-L-lysine can be replaced with another polylysine or with polyethylene glycol, a poly-L-ornithine, a poly-L-arginine or a poly-L-histidine.

Preferentially, the stirring is carried out for at least 60 minutes, even more preferentially between 5 and 20 minutes, and preferentially at a stirring speed of 900 revolutions per minute or less, in particular at a stirring speed of between 50 and 800 revolutions per minute.

According to one preferred embodiment, the production method according to the invention also comprises a step c. of separating the soluble and insoluble phases, in order to recover the soluble phase. In this case, the insoluble phase is removed and the soluble phase constitutes composition C1 according to the invention. Specifically, a physical separation process (filtration, ultrafiltration) is preferentially carried out to ensure the isolation of the soluble fraction containing both the amphiphilic premix and the solubilized molecules.

Composition C1 in liquid form can then be freeze-dried or dehydrated to be in solid form, preferentially by means of slow freeze-drying, for example between 12 and 36 hours.

Moreover, if active molecule-polymer conjugates of composition C1 are not incorporated into the premix in step a, they can be added to the mixture after step b., i.e. after the formation of micelles and the encapsulation of farnesyl cysteine and cholesterol.

The active molecule-polymer conjugates can be produced by any means known to a person skilled in the art for conjugating a molecule covalently to a polymer depending on their chemical nature. Thus, lauric acid, myristic acid, palmitic acid, oleic acid, linoleic acid, orotic acid, azelaic acid, thioctic acid, acetic acid, palmitoleic acid, butyric acid, lactic acid, propionic acid and oleic acid are conjugated to the polymer (preferentially poly-L-lysine) via an amide bond.

Exemplary embodiments of amide, urea and carbamate bonds are described, for example, in:
Ryser HJ, Shen WC. Conjugation of methotrexate to poly (L-lysine) as a potential way to overcome drug resistance. Cancer. 1980 Mar. 15;45(5 Suppl): 1207-11 (amide bonds),
Zhuxian Z, Jianbin T, Qihang S, William J. A multifunctional PEG-PLL drug conjugate forming redox-responsive nanoparticles for intracellular drug delivery, Issue 38, 2015. Journal of Materials Chemistry B (amide bonds),
Scheper V, Wolf M, Scholl M, Kadlecova Z, Perrier T, Klok HA, Saulnier P, Lenarz T, Stover T. Potential novel drug carriers for inner ear treatment: hyperbranched polylysine and lipid nanocapsules. Na-nomedicine (Lond). 2009 Aug;4 (6): 623-35 (urea bonds),
Stéphanie Gac-Breton, Jean Coudane, Mahfoud Boustta & Michel Vert (2004) Norfloxacin-Poly (l-Lysine Citramide Imide) Conjugates and Structure-dependence of the Drug Release, Journal of Drug Targeting, 12:5, 297-307, (carbamate bonds),
Elmore, W. M. (2013). Nanoparticles Stabilized with MPEG-Polylysine Carbamate: Synthesis and Characterization, (carbamate bonds),
Ning-Ping Huang, Janos Voros, Susan M. De Paul, Marcus Textor, and Nicholas D. Spencer. Biotin-Derivatized Poly (l-lysine)-g-poly (ethylene glycol): A Novel Polymeric Interface for Bioaffinity Sensing. Langmuir 2002 18 (1), 220-230 (carbamate bonds).

### Use of Composition C1

Composition C1 is a generic formulation particularly suitable for preventing and/or treating multifactorial diseases, in particular multifactorial chronic diseases, and in particular diseases involving intestinal permeability.

It is capable of acting in a combined manner:
as an immunomodulator and autoimmune regulator, in particular by virtue of the presence of oleic acid, palmitic acid, palmitoleic acid,
as an anti-inflammatory, in particular by virtue of the presence of palmitic acid, azelaic acid, farnesyl cysteine, palmitoleic acid, myristic acid,
as an antibacterial, in particular by virtue of the presence of lauric acid, azelaic acid,
as an antifungal, in particular by virtue of the presence of lauric acid,
as an antiradical, in particular by virtue of the presence of farnesyl cysteine,
as an antagonist of isoprenylated proteins, in particular by virtue of the presence of farnesyl cysteine,
on the stabilization of cell membranes, in particular by virtue of the presence of cholesterol,
as a scavenger of free radicals and of metals, in particular by virtue of the presence of thioctic acid.
as an antiviral, in particular by virtue of the presence of myristic acid,
as a synergistic cell substrate, in particular by virtue of the presence of butyrate,
to promote the development of intestinal flora, in particular by virtue of the presence of orotic acid,
to regulate mucous membranes and intestinal flora, in particular by virtue of the presence of acetate, lactate, propionate and butyrate

The various molecules present act synergistically and in particular make it possible to restore the function of intestinal permeability.

Thus, composition C1 can be used to restore the function of the intestinal membrane and/or to maintain intestinal permeability and/or to prevent or combat intestinal mucosa hyperpermeability.

According to one variant, the invention relates to the use of composition C1 as a food supplement in a healthy person or healthy animal, in particular to reduce the intestinal permeability of the person or animal to whom or which it is given.

The intestine therefore plays a key role in the initiation and regulation of the chronicity of diseases. Thus, composition C1 can in particular be administered to humans or animals to prevent or combat diseases in which the intestine plays an important role, in particular chronic diseases, and in particular degenerative chronic diseases, such as neurodegenerative diseases like amyotrophic lateral sclerosis. Composition C1 can also be used as an immune system activator to prevent or combat autoimmune and infectious diseases.

The composition can be used in particular to reduce the chronicity of chronic diseases.

Preferentially, composition C1 is used in prevention or treatment as soon as the first symptoms of intestinal hyperpermeability or of a chronic disease appear, in order to prevent the development of the chronicity of said disease and thereby prevent the cytokine storm which results from prolonged intestinal hyperpermeability.

Specifically, composition C1 is particularly useful for at least:
reducing the inflammatory process, and/or
reducing the autoimmune process, and/or
reducing intestinal permeability, and/or
an antioxidant effect, and/or
an antiradical effect, and/or
an antibacterial and/or antiviral/or antifungal effect.

The invention therefore also relates to a composition C1 according to the invention for its use as a drug, in particular in humans or animals, i.e. for a human or animal subject.

In particular, the invention relates to a composition C1 for use thereof in the prevention and/or treatment of a multifactorial disease, in particular a chronic multifactorial disease, in particular a disease selected from among inflammatory diseases, neurodegenerative diseases, bacterial diseases, viral diseases, autoimmune diseases, allergies and food intolerances.

According to one particular embodiment, the invention relates to a composition C1 for use thereof in the prevention and/or treatment of a disease selected from among eczema, psoriasis, chronic inflammatory bowel diseases (in particular Crohn's disease and celiac disease), Alzheimer's disease, Parkinson's disease, multiple sclerosis, and amyotrophic lateral sclerosis.

Composition C1 can be used alone or with one or more other compositions.

According to one embodiment, composition C1 is preferentially used with a composition C2.

### Composition C2

The present invention therefore also relates to a composition C2 comprising at least the following active molecules:
taurine,
cysteine,
methionine
spermine
retinoic acid,
pantothenic acid,
biotin,
coenzyme Q10,
glutathione,
ascorbic acid,
GABA,
alpha-tocopherol,
   and/or a salt and/or an ester and/or an anhydride of one or more of these molecules.

For all of the active molecules present in the compositions according to the invention, they can be the molecules per se (e.g. ascorbic acid) and/or a salt of these molecules (e.g. ascorbate) and/or an ester of these molecules (e.g. ascorbic acid ester) and/or an anhydride (e.g. ascorbic acid anhydride).

Preferentially:
the amount of retinoic acid in composition C2 is greater than that of each of the other molecules taken individually, and/or
the amount of taurine, the amount of methionine and the amount of GABA are equal in composition C2 and are each greater than that of each of the other molecules taken individually, except for retinoic acid and coenzyme Q10.

Preferentially, each of the active molecules in composition C2 represents between 6 E-05 M and 18 E-05 M.

According to one embodiment, in order to improve the solubility, efficacy and bioavailability of the molecules of composition C2, at least one of the molecules of the composition selected from among taurine, spermine, pantothenic acid, biotin, glutathione, ascorbic acid, GABA, and alpha-tocopherol, salts of these molecules, esters of these molecules, and anhydrides of these molecules is covalently conjugated to at least one molecule of a polymer selected from among polylysine, polyethylene glycol, polyornithine, polyarginine and polyhistidine.

Specifically, the molecules selected from among taurine, spermine, pantothenic acid, biotin, retinoic acid, glutathione, ascorbic acid, GABA, and alpha-tocopherol, salts of these molecules, esters of these molecules and anhydrides of these molecules can all be covalently conjugated to a polymer selected from among polylysine, polyethylene glycol, polyornithine, polyarginine and polyhistidine, in particular via an amide, urea or carbamate bond.

According to one particularly suitable variant, all of the molecules selected from among taurine, spermine, retinoic acid, pantothenic acid, biotin, glutathione, ascorbic acid, GABA, and alpha-tocopherol, salts of these molecules, esters of these molecules and anhydrides of these molecules, present in composition C1 according to invention, are covalently conjugated to at least one molecule of a polymer selected from among polylysine, polyethylene glycol, polyornithine, polyarginine and polyhistidine.

Cysteine and methionine, and esters, salts and anhydrides of cysteine or methionine, cannot be covalently bonded, i.e. cannot be conjugated, to a polymer selected from among polylysine, polyethylene glycol, polyornithine, polyarginine and polyhistidine. Thus, preferentially, cysteine and/or methionine, and/or esters and/or salts thereof, present in composition C2 are bonded to said polymer by copolymerization to form a copolymer. Preferentially, cysteine and/or methionine, and/or esters and/or salts and/or anhydrides thereof, are included in the polypeptide backbone of the polymer to form a copolymer.

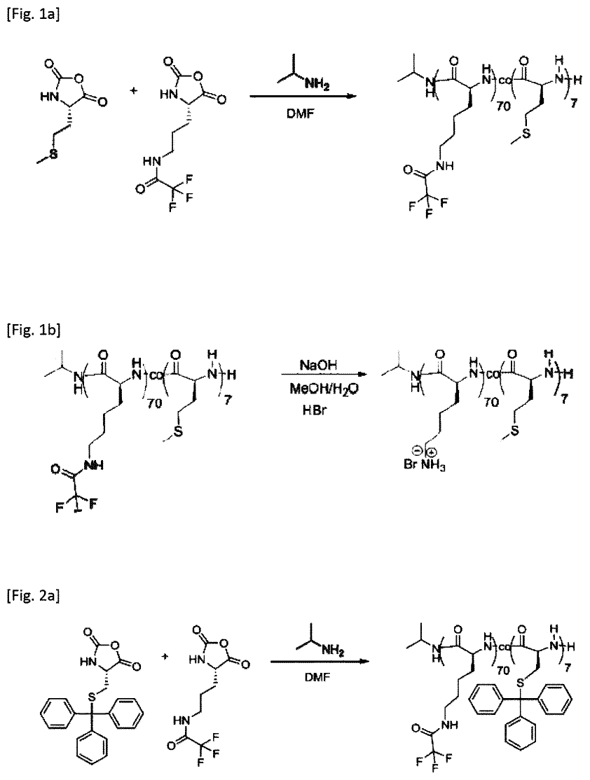

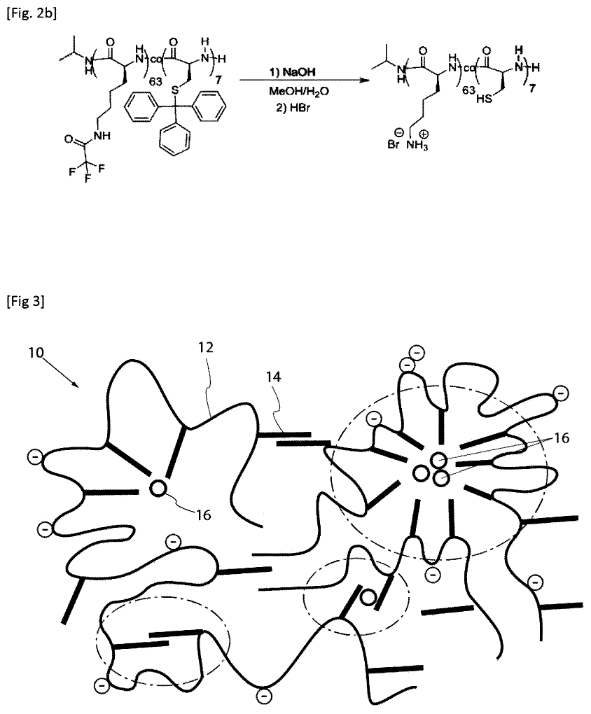

The copolymerization can be carried out in particular by the implementing a step of polymerization (for example L-Met NCA or L-Cys NCA and L-Lys NCA are mixed and polymerized to obtain a copolymer) followed by a step of deprotecting the copolymer obtained after polymerization. Examples are presented in FIGS. 1a, 1b (poly-(L-lysine-co-methionine), 2a and 2b (poly-(L-lysine-co-cysteine):
1a: Polymerization: The L-Met NCA and L-Lys NCA (trifluoroacetate) are mixed and polymerized in the appropriate ratio to obtain the appropriate random copolymer,
1b: Deprotection of PLys-TFA: The preceding copolymer is deprotected to obtain PLys in the form of a bromide salt.
2a: Polymerization: The L-Cys NCA (trityl) and L-Lys NCA (trifluoroacetate) are mixed in the appropriate ratio to obtain the appropriate random copolymer.
2b: Deprotection of Lys-TFA: The preceding copolymer is deprotected to obtain L-Lys in the form of a bromide salt and L-Cys in its deprotected form.

Thus, in composition C2, cysteine and methionine are each conjugated to a molecule of a polymer selected from among polylysine, polyethylene glycol, polyornithine, polyarginine and polyhistidine by polymerization, said molecule and said polymer forming a copolymer. Preferentially, the copolymers comprise between 5 and 20% cysteine or methionine and between 80 and 95% polymer (selected from among polylysine, polyethylene glycol, polyornithine, polyarginine and polyhistidine). According to one preferred variant, the copolymers comprise 10% cysteine or methionine and 90% polymer (ratio of 1 to 10).

Among all of the molecules to be conjugated, one of them is highly hydrophobic (logP values greater than 1, preferentially greater than 2 and preferentially greater than 3) and is chemically not suitable for covalent conjugation due to the absence of reactive functional groups and due to chemical incompatibility. This is coenzyme Q10. However, given both the hydrophilic character of certain polymers (such as polylysine, polyethylene glycol, polyornithine, polyarginine or polyhistidine) and the hydrophobic character of certain molecules suitable for covalent conjugation (alpha-tocopherol, retinoic acid), then, according to the invention, these particular conjugates behave as surfactants in aqueous solution. In this way, some of these (amphiphilic) conjugates act as polymeric micelles in an aqueous medium, creating a hydrophobic pocket capable of encapsulating hydrophobic coenzyme Q10, thereby considerably improving the solubility and stability in aqueous solution thereof.

Specifically, coenzyme Q10 and esters, salts and anhydrides thereof cannot be covalently bonded, i.e. cannot be conjugated, to a polymer selected from among polylysine, polyethylene glycol, polyornithine, polyarginine and polyhistidine. Thus, preferentially, coenzyme Q10 and/or esters and salts of coenzyme Q10 are encapsulated in micelles. Thus, composition C2 preferentially comprises micelles in which at least coenzyme Q10 and/or esters and/or salts and/or anhydrides of coenzyme Q10 are encapsulated.

According to one particularly suitable embodiment, at least one of the micelles is formed by amphiphilic conjugates each consisting of at least one hydrophobic molecule covalently conjugated to a molecule of a polymer selected from among polylysine, polyethylene glycol, polyornithine, polyarginine and polyhistidine. Even more preferably, as shown in FIG. 3, at least one of the micelles 10 is formed by amphiphilic conjugates each consisting of at least one molecule 14 selected from among taurine, spermine, pantothenic acid, biotin, glutathione, ascorbic acid, GABA, and alpha-tocopherol, salts of these molecules, esters of these molecules, and anhydrides of these molecules covalently conjugated to a molecule of a polymer 12 selected from among polylysine, polyethylene glycol, polyornithine, polyarginine and polyhistidine, encapsulating coenzyme Q10 and/or esters and/or salts and/or anhydrides of coenzyme Q10 16. Specifically, taurine, spermine, pantothenic acid, biotin, glutathione, ascorbic acid, GABA, and alpha-tocopherol, salts of these molecules, esters of these molecules and anhydrides of these molecules, are hydrophobic and poly-L-lysine, polyethylene glycol, polyornithine, polyarginine and polyhistidine are hydrophilic polymers.

Thus, composition C2 preferentially comprises:
conjugates of taurine, retinoic acid, spermine, pantothenic acid, biotin, glutathione, ascorbic acid, GABA, and alpha-tocopherol, salts of these molecules, esters of these molecules and/or anhydrides of these molecules, with polylysine, polyethylene glycol, polyornithine, polyarginine or polyhistidine, and
micelles formed by at least one of these conjugates, said micelles encapsulating coenzyme Q10, and
copolymers of methionine and of polylysine, polyethylene glycol, polyornithine, polyarginine or polyhistidine, and/or copolymers of cysteine and of polylysine, polyethylene glycol, polyornithine, polyarginine or polyhistidine.

In particular, this configuration makes it possible:
to increase the half-life of the active molecules of the composition in the body,
to target tissues or cells on which the molecules of the composition are to act,
to allow the active molecules of the composition to cross the blood-brain barrier and to enter cells
to increase the stability and bioavailability of the active ingredient.

The efficacy of composition C2 is therefore enhanced and it is possible to administer lower doses and reduce the acute or chronic toxicity of the active molecules contained in the composition.

Preferentially, the one or more polymers conjugated to the molecules or with which the molecules are copolymerized are selected from among poly-L-lysine, polyethylene glycol, poly-L-ornithine, poly-L-arginine and poly-L-histidine.

According to one embodiment, it is polylysine, preferentially poly-L-lysine. Preferentially, the polylysine used is a poly-L-lysine with a molecular weight of between 12000 and 20000 Da.

Thus, according to one variant, composition C2 comprises at least:
A. the following conjugates, each conjugate consisting of a molecule covalently bonded to a polylysine:
one or more taurine-poly-L-lysine conjugates
one or more spermine-poly-L-lysine conjugates
one or more pantothenyl-poly-L-lysine conjugates
one or more alpha-tocopherol-poly-L-lysine conjugates
one or more biotinyl-poly-L-lysine conjugates
one or more glutathione-poly-L-lysine conjugates
one or more ascorbyl-poly-L-lysine conjugates
one or more GABA-poly-L-lysine conjugates,
one or more retinoyl-poly-L-lysine conjugates, and
B. coenzyme Q10, and/or an ester and/or a salt of coenzyme Q10, encapsulated in micelles, preferentially in micelles formed by one or more of the conjugates listed under point A, and
C. methionine-poly-L-lysine copolymers and cysteine-poly-L-lysine copolymers, where the methionine and/or cysteine can be replaced with an ester or a salt of these molecules.

In this composition, the poly-L-lysine can be replaced with another polylysine or with polyethylene glycol, a poly-L-ornithine, a poly-L-arginine or a poly-L-histidine.

Composition C2 according to the invention can be in solid form or in liquid form. When in liquid form, the composition comprises at least water and the constituents mentioned above. The solid form is preferentially obtained from the liquid form, preferentially by freeze-drying. Thus, when composition C2 in liquid form comprises micelles and it is freeze-dried into solid form, the micelles reform when composition C2 in solid form is placed back into an aqueous solution.

Composition C2 also preferentially comprises pharmaceutically acceptable excipients. These excipients can be selected in particular to meet the pH and osmolarity requirements of solutions for injection into humans or animals. For example, they can be acids or bases for adjusting pH or NaCl for adjusting osmolarity.

Composition C2 according to the invention is intended to be administered to human beings, or to an animal, and is consequently in a form suitable for such administration. When it is in liquid form, it is preferentially suitable for subcutaneous or intravenous administration, in particular intravenous infusion, and it is packaged in suitable containers known to a person skilled in the art for packaging this type of product. Composition C2 according to the invention can also be administered in liquid form via a pump, like an insulin pump.

When it is in solid form, it is preferentially suitable for administration:
transcutaneously and it is preferentially formulated and packaged in the form of a patch, or
nasally in the form of a powder, or
sublingually in the form of a powder or tablet, or
transmucosally, and it is preferentially formulated and packaged in the form of a mucoadhesive tablet.

### Method for Producing Composition C2

Composition C2 according to the invention can be produced using any suitable method.

If the molecules that constitute composition C2 are used as they are in a solvent, they can all be mixed together in said solvent.

If the molecules in composition C2 are conjugated to polymers for some, copolymerized for others and in micelles for others, the production method can comprise the following steps:
a. creating an amphiphilic premix: mixing, in an aqueous solution, one or more conjugates selected from among:
one or more taurine-poly-L-lysine conjugates
one or more spermine-poly-L-lysine conjugates
one or more pantothenyl-poly-L-lysine conjugates
one or more alpha-tocopherol-poly-L-lysine conjugates
one or more biotinyl-poly-L-lysine conjugates
one or more glutathione-poly-L-lysine conjugates
one or more ascorbyl-poly-L-lysine conjugates
one or more GABA-poly-L-lysine conjugates,
one or more retinoyl-poly-L-lysine conjugates, and
b. adding at least coenzyme Q10 to the amphiphilic premix and stirring so as to form micelles formed by one or more of the conjugates of the amphiphilic premix encapsulating the coenzyme Q10,
adding methionine-poly-L-lysine and cysteine-poly-L-lysine copolymers to the mixture.

Specifically, one variant of the invention consists in taking advantage of the amphiphilic nature of the majority of the individual components to create an amphiphilic premix which allows the controlled solubilization of the hydrophobic coenzyme Q10. The solubilization process according to one preferred embodiment consists in the controlled addition of the hydrophobic molecules to the amphiphilic premixes and allowing the time needed for their dissolution with stirring.

The poly-L-lysine can be replaced with another polylysine or with polyethylene glycol, a poly-L-ornithine, a poly-L-arginine or a poly-L-histidine.

Preferentially, the stirring is carried out for at least 5 minutes, even more preferentially between 5 and 20 minutes, and preferentially at a stirring speed of 900 revolutions per minute or less, in particular at a stirring speed of between 50 and 800 revolutions per minute.

According to one preferred embodiment, the production method according to the invention also comprises a step d. of separating the soluble and insoluble phases, in order to recover the soluble phase. In this case, the insoluble phase is removed and the soluble phase constitutes composition C2 according to the invention. Specifically, a physical separation process (filtration, ultrafiltration) is preferentially carried out to ensure the isolation of the soluble fraction containing the amphiphilic premix, the copolymers and the solubilized molecules.

Composition C2 in liquid form can then be freeze-dried or dehydrated to be in solid form, preferentially by means of slow freeze-drying, for example between 12 and 36 hours.

Moreover, if active molecule-polymer conjugates and/or the copolymers of the composition are not incorporated into the premix in step a or c, they can be added to the mixture after step b., i.e. after the formation of micelles and the encapsulation of coenzyme Q10.

The active molecule-polymer conjugates can be produced by any means known to a person skilled in the art for conjugating a molecule covalently to a polymer depending on their chemical nature. Thus:
ascorbic acid is conjugated to a polymer via a carbamate bond,
spermine and taurine are conjugated to a polymer via a urea bond.
glutathione, pantothenic acid, aminobutyric acid, biotin, alpha tocopherol and GABA, and retinoic acid are conjugated to a polymer via an amide bond.

Exemplary embodiments of urea and carbamate bonds are described, for example, in:
Zhuxian Z, Jianbin T, Qihang S, William J. A multifunctional PEG-PLL drug conjugate forming redox-responsive nanoparticles for intracellular drug delivery, Issue 38, 2015. Journal of Materials Chemistry B (amide bonds),
Scheper V, Wolf M, Scholl M, Kadlecova Z, Perrier T, Klok HA, Saulnier P, Lenarz T, Stover T. Potential novel drug carriers for inner ear treatment: hyperbranched polylysine and lipid nanocapsules. Na-nomedicine (Lond). 2009 Aug;4 (6): 623-35 (urea bonds),
Stephanie Gac-Breton, Jean Coudane, Mahfoud Boustta & Michel Vert (2004) Norfloxacin-Poly (l-Lysine Citramide Imide) Conjugates and Structure-dependence of the Drug Release, Journal of Drug Targeting, 12:5, 297-307, (carbamate bonds),
Elmore, W. M. (2013). Nanoparticles Stabilized with MPEG-Polylysine Carbamate: Synthesis and Characterization, (carbamate bonds),
Ning-Ping Huang, Janos Voros, Susan M. De Paul, Marcus Textor, and Nicholas D. Spencer. Biotin-Derivatized Poly (l-lysine)-g-poly (ethylene glycol): A Novel Polymeric Interface for Bioaffinity Sensing. Langmuir 2002 18 (1), 220-230 (carbamate bonds).

The copolymers can be produced by any means known to a person skilled in the art. The methionine or cysteine molecules are incorporated into the structure of the polymer preferentially in the presence of an anhydrous solvent and, according to one embodiment, in a ratio of 1 cysteine or methionine molecule to 10 monomers of the polymer.

### Use of Composition C2

Composition C2 is a formulation particularly suitable for preventing and/or treating multifactorial diseases, in particular multifactorial chronic diseases. Composition C2 is particularly useful for the prevention and treatment of neurodegenerative, autoimmune or infectious diseases, or cancers. In particular, it has great efficacy in the prevention and treatment of amyotrophic lateral sclerosis. Composition C2 intervenes in conventional and known processes to reduce the chronicity of diseases. Its action is particularly suited to neurodegenerative diseases, autoimmune diseases, cancer and infectious diseases.

In particular, it is capable of acting in a combined manner:
as an immunomodulator and autoimmune regulator, in particular by virtue of the presence of retinoic acid and GABA,
as a neuroprotector, in particular by virtue of the presence of taurine, coenzyme Q10, GABA
as an anti-inflammatory, in particular by virtue of the presence of spermine,
as cell protector, in particular by virtue of the presence of pantothenic acid, coenzyme Q10 (protection at the mitochondrial chain level in particular)
as a cell membrane protector, in particular by virtue of the presence of a-tocopherol,
as an antioxidant, in particular by virtue of the presence of taurine, cysteine, methionine, spermine, pantothenic acid, alpha-tocopherol, coenzyme Q10, glutathione, ascorbic acid,
as a cell metabolism regulator scavenging reactive oxygen species, in particular by virtue of ascorbic acid,
as a glutathione precursor, in particular by virtue of the presence of cysteine,
on the stabilization of the cell membranes,
as a scavenger of free radicals and metals, in particular by virtue of the presence of taurine, cysteine, methionine, spermine, pantothenic acid, coenzyme Q10, glutathione.

The various molecules present act synergistically and in particular make it possible to prevent or combat factors that are at the root of chronic diseases and amyotrophic lateral sclerosis in particular.

Thus, composition C2 can be used to prevent and/or combat chronic diseases, in particular to act on disease-specific metabolisms. Composition C2 can be used in particular to reduce the chronicity of chronic diseases.

Preferentially, composition C2 is used in prevention or treatment as soon as the first symptoms of a chronic disease, in particular a neurodegenerative disease and specifically amyotrophic lateral sclerosis, appear.

The invention therefore also relates to a composition C2 according to the invention for its use as a drug, in particular in humans or animals, i.e. for a human or animal subject.

In particular, the invention relates to a composition C2 for use thereof in the prevention and/or treatment of a multifactorial disease, in particular a chronic multifactorial disease, in particular a disease selected from among inflammatory diseases, neurodegenerative diseases, bacterial diseases, viral diseases, autoimmune diseases, allergies and food intolerances, and in particular amyotrophic lateral sclerosis.

According to one particular embodiment, the invention relates to a composition C1 for use thereof in the prevention and/or treatment of a disease selected from among eczema, psoriasis, chronic inflammatory bowel diseases (in particular Crohn's disease and celiac disease), Alzheimer's disease, Parkinson's disease, multiple sclerosis, and amyotrophic lateral sclerosis.

Composition C2 is very preferentially used with composition C1.

One particular subject of the invention is therefore composition C1 for use thereof with composition C2 as a drug, in particular in the prevention and/or treatment of a multifactorial disease, in particular a chronic multifactorial disease, in particular a disease selected from among inflammatory diseases, neurodegenerative diseases, bacterial diseases, viral diseases, autoimmune diseases, allergies and food intolerances, and in particular in the prevention and/or treatment of a disease selected from among eczema, psoriasis, Crohn's disease, celiac disease, chronic inflammatory bowel diseases, Alzheimer's disease, Parkinson's disease, multiple sclerosis, and amyotrophic lateral sclerosis.

Composition C1 and composition C2 are used a drug simultaneously or with a time gap between them. In particular, it is preferable to administer composition C1 first, followed by composition C2, the two administrations preferentially being separated by at least 1 hour, even more preferentially by at least 4 hours, ideally one in the morning and one in the evening.

Compositions C1 and C2 can also be used to treat or prevent diseases associated with pathological dysbiosis of the intestinal microbiota. In particular, compositions C1 and C2 can be used to prevent or treat: a neurodegenerative disease associated with pathological dysbiosis of the intestinal microbiota, for example a disease selected from among amyotrophic lateral sclerosis, multiple sclerosis, Parkinson's disease, and Alzheimer's disease an intestinal disease associated with pathological dysbiosis of the intestinal microbiota, for example a disease selected from among Crohn's disease, chronic inflammatory bowel diseases, hemorrhagic rectocolitis, irritable bowel syndrome, ulcerative colitis, rheumatoid arthritis and food intolerance, in particular gluten intolerance.

The invention is now described with the aid of examples and test results.

### EXAMPLES

### Example 1: Composition C1

An exemplary composition C1 suitable for testing on mice is presented below.

The following amounts of conjugates were weighed according to table 1 in order to prepare a stock of 300 ml of composition C1 10×. All of the conjugates were weighed using an analytical balance under a laminar flow hood.

TABLE 1

| PLL conjugate | |
|---|---|
| (PLL = poly-L-lysine) | Amount (mg) |
| Oleyl-PLL | 201 |
| Palmitic-PLL | 78 |
| Lauryl-PLL | 189 |
| Lynoleyl-PLL | 78 |
| Azelayl-PLL | 75 |
| Palmitoleyl-PLL | 156 |
| Thioctyl-PLL | 225 |
| Myristyl-PLL | 78 |
| Orotyl-PLL | 72 |
| Acetate-PLL | 174 |
| Buyrate-PLL | 285 |
| Lactate-PLL | 177 |
| Propionate-PLL | 177 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

In parallel, cholesterol and farnesyl cysteine molecules were dissolved separately at the approximate concentration of 50 mg/ml in absolute ethanol and filtered over a 0.22 μm filter. The solvent was evaporated using a rotary evaporator. The required amounts of dry solids were weighed under a laminar flow hood, as presented in table 2.

TABLE 2

| Molecule | Amount (mg) |
|---|---|
| Cholesterol | 12 |
| Farnesyl cysteine | 12 |

In a sterile 500 ml flask equipped with a magnetic bar, 23.8 ml of each conjugated solution prepared previously was added while being magnetically stirred (700 revolutions per minute).

The corresponding amounts of (previously freeze-dried) salts at 300 of PBS were added to the solution of conjugates while being magnetically stirred (700 revolutions per minute).

Cholesterol and farnesyl cysteine were added and the mixture was stirred for 25 hours (700 revolutions per minute).

30 ml of formulation C1 10× was taken under a laminar flow hood and added to a new sterile 500 ml flask.

270 ml of sterile PBS was added to obtain formulation C1 1×. The formulation was stirred for 30 minutes.

The flasks were prepared according to the following plan using a sterile calibrated 5 ml pipette: C1 1×=83 flasks, 2 ml each. C1 10×=83 flasks, 2 ml each.

The 166 flasks were placed in a steel freeze-drying unit and a freeze-drying cycle of one day was initiated.

### Example 2: Composition C2

An exemplary composition C2 suitable for testing on mice is presented below.

The following amounts of conjugates were weighed according to table 3 in order to prepare a stock of 300 ml of composition C2 10×. All of the conjugates were weighed using an analytical balance under a laminar flow hood.

TABLE 3

| PLL conjugate | |
|---|---|
| (PLL = poly-L-lysine) | Amount (mg) |
| Alpha-tocopherol-PLL | 210 |
| Ascorbyl PLL | 189 |
| Biotinyl-PLL | 204 |
| GABA-PLL | 378 |
| Glutathione-PLL | 222 |
| Pantothenyl-PLL | 204 |
| Retinoyl-PLL | 606 |
| Spermine-PLL | 198 |
| Taurine-PLL | 330 |
| PLL/Cysteine (copolymer) | 180 |
| PLL/Methionine (copolymer) | 303 |

Each conjugate was weighed in a sterile 50 ml centrifuge tube and was stored in the freezer at −20° C. until use.

In parallel, coenzyme Q10 was dissolved at the approximate concentration of 50 mg/ml in absolute ethanol and filtered through a 0.22 μm filter. The solvent was evaporated using a rotary evaporator. The required amounts of dry solids were weighed under a laminar flow hood, as presented in table 4.

TABLE 4

| Molecule | Amount (mg) |
|---|---|
| Coenzyme Q10 | 156 |

The solid was stored in a sterile centrifuge tube at −20° C. until use.

The 50 ml centrifuge tubes previously prepared with the conjugates and copolymers were taken and left to warm up to room temperature. After this time, 30 ml of water was added to each centrifuge tube.

The conjugates and copolymers were dissolved using a vortex mixer, stirring for a time of between 10 and 20 minutes depending on the solubility of the conjugates and copolymers.

The centrifuge tube containing coenzyme Q10 was also taken from the freezer and allowed to warm up to room temperature.

A sterile 500 ml flask was taken and provided with a magnetic bar. 27.3 ml of each conjugated solution prepared previously was added to the flask while being magnetically stirred (700 revolutions per minute).

The corresponding amounts of (previously freeze-dried) salts at 300 of PBS were added to the solution of conjugates while being magnetically stirred (700 revolutions per minute).

Coenzyme Q10 was added and the mixture was stirred for 2 hours (700 revolutions per minute).

After 2 hours, 30 ml of formulation C2 10× was taken under a laminar flow hood and added to a new sterile 500 ml flask.

270 ml of sterile PBS was added to obtain formulation C2 X1. The formulation was stirred for 30 minutes.

The flasks were prepared according to the following plan using a sterile calibrated 5 ml pipette: DP2 1×=83 flasks, 2 ml each. DP2 10×=83 flasks, 2 ml each.

The 166 flasks were placed in the steel freeze-drying unit and a freeze-drying cycle of one day was initiated.

### Example 3: Composition C1

An exemplary composition C1 suitable for testing on rats is presented below.

The method for producing the composition is similar to that presented in example 1. The concentrations of each of the constituents are different and presented in table 5.

TABLE 5

| | Concentration | Concentration |
|---|---|---|
| Components | C1 10X (mg/mL) | C1 1X (mg/mL) |
| Acetate-PLL | 1.16 | 0.12 |
| Azelayl-PLL | 0.50 | 0.05 |
| Buyrate-PLL | 1.88 | 0.19 |
| Lauryl-PLL | 1.26 | 0.13 |
| Lactate-PLL | 1.18 | 0.12 |
| Linoleyl-PLL | 0.52 | 0.05 |
| Myristyl-PLL | 0.51 | 0.05 |
| Oleyl-PLL | 1.33 | 0.13 |
| Orotyl-PLL | 0.48 | 0.05 |
| Palmitic-PLL | 0.52 | 0.05 |
| Palmitoleyl-PLL | 1.04 | 0.10 |
| Propionate-PLL | 1.17 | 0.12 |
| Thioctyl-PLL | 1.49 | 0.15 |
| Cholesterol | 0.09 | 0.01 |
| FarCys | 0.07 | 0.01 |

The sum of the concentrations of the conjugates PLL+cholesterol+farnesyl cysteine for C1 10× is 13.2 mg/mL.

The sum of the concentrations of the conjugates PLL+cholesterol+farnesyl cysteine for C1 1× is 1.32 mg/ml.

### Example 4: Composition C1

An exemplary composition C1 suitable for humans is presented below.

The method for producing the composition is similar to that presented in example 1. The concentrations of each of the constituents are different and presented in table 6.

TABLE 6

| | Concentration | Concentration |
|---|---|---|
| Components | C1 10X (mg/mL) | C1 1X (mg/mL) |
| Acetate-PLL | 7.192 | 0.744 |
| Azelayl-PLL | 3.1 | 0.31 |
| Buyrate-PLL | 11.656 | 1.178 |
| Lauryl-PLL | 7.812 | 0.806 |
| Lactate-PLL | 7.316 | 0.744 |
| Linoleyl-PLL | 3.224 | 0.31 |
| Myristyl-PLL | 3.162 | 0.31 |
| Oleyl-PLL | 8.246 | 0.806 |
| Orotyl-PLL | 2.976 | 0.31 |
| Palmitic-PLL | 3.224 | 0.31 |
| Palmitoleyl-PLL | 6.448 | 0.62 |
| Propionate-PLL | 7.254 | 0.744 |
| Thioctyl-PLL | 9.238 | 0.93 |
| Cholesterol | 0.558 | 0.062 |
| FarCys | 7.192 | 0.744 |

### Example 5: Composition C2

An exemplary composition C2 suitable for humans is presented below.

The method for producing the composition is similar to that presented in example 2. The concentrations of each of the constituents are different and presented in table 7.

TABLE 7

| | Concentration | Concentration |
|---|---|---|
| Components | C1 10X (mg/mL) | C1 1X (mg/mL) |
| Alpha-tocopherol-PLL | 8.61 | 0.861 |
| Ascorbyl PLL | 7.749 | 0.7749 |
| Biotinyl-PLL | 8.364 | 0.8364 |
| GABA-PLL | 15.498 | 1.5498 |
| Glutathione-PLL | 9.102 | 0.9102 |
| Pantothenyl-PLL | 8.364 | 0.8364 |
| Retinoyl-PLL | 24.846 | 2.4846 |
| Spermine-PLL | 8.118 | 0.8118 |
| Taurine-PLL | 13.53 | 1.353 |
| PLL/Cysteine (copolymer) | 7.38 | 0.738 |
| PLL/Methionine (copolymer) | 12.423 | 1.2423 |
| Coenzyme Q10 | 6.396 | 0.6396 |

### Test Results

### Study on the Effect of Composition C1 According to the Invention on Restoring Intestinal Permeability

The purpose of this study is to evaluate the efficacy of an injectable formulation according to the invention on the restoration of intestinal permeability following the induction of intestinal inflammation caused by dextran sulfate sodium (DSS) in rats.

The intestinal epithelium is a dynamic barrier that limits the access of bacteria, parasites, viruses and also certain chemical molecules to the tissues of the host. It is composed of different types of cells, each having a well-defined role, such as enterocytes which are responsible for nutrient absorption, goblet cells which secrete mucus, the thickness of which keeps bacteria and other microorganisms away from the enterocytes, Paneth cells which are located at the bottom of the crypts of the small intestine, and participate in maintaining homeostasis and in defending the intestinal mucosa via the secretion of antimicrobial agents such as defensins, and microfold cells, or M cells, which specialize in the endocytosis of antigens, molecules or microorganisms present in the intestinal lumen. They act as antigen-presenting cells since they present endocytized antigens to the underlying immune system. The epithelial physical barrier provided by the enterocytes is reinforced by a layer of glycocalyx and of thick mucus (this layer is thinner at M cells for easier access to the microbiome of the intestinal lumen) and secretory IgA via enterocyte transcytosis produced by the underlying plasmocytes. The epithelial cells form a monolayer of polarized cells resting on the lamina propria via their basolateral poles and they are closely connected together by tight junctions. Peyer's patches are located in this lamina propria, these consisting of follicles rich in lymphocytes and are where pro-or anti-inflammatory responses are initiated. Many diseases are associated with a change in intestinal permeability, including chronic inflammatory bowel diseases, obesity, multiple sclerosis, amyotrophic lateral sclerosis and Alzheimer's disease. This change leads to an increase in intestinal permeability usually resulting in inflammation of the intestine wall.

The composition tested in this study is composition C1 from example 3.

The intestinal epithelium is a dynamic barrier that limits the access of bacteria, parasites, viruses and also certain chemical molecules to the tissues of the host. It is composed of different types of cells, each having a well-defined role, such as enterocytes which responsible for the absorption function of the intestine, goblet cells which secrete mucus, M cells which are responsible for transporting macromolecules and antigens from the intestinal lumen (lamina propria) to underlying immune cells and neuroendocrine cells. The epithelial physical barrier provided by the enterocytes is reinforced by a layer of glycocalyx and of thick mucus. The epithelial cells form a monolayer of polarized cells resting on the lamina propria via their basolateral poles and they are closely connected together by tight junctions. Many diseases are associated with a change in intestinal permeability, including chronic inflammatory bowel diseases, obesity, multiple sclerosis, amyotrophic lateral sclerosis and Alzheimer's disease. This change leads to an increase in intestinal permeability usually resulting in inflammation of the intestine wall.

The main objective of the study is to demonstrate the efficacy of the invention on the remediation of intestinal permeability following the induction of chronic intestinal inflammation.

To test these hypotheses, rats received different doses of composition C1 from example 3, administered by subcutaneous injection after induction of intestinal inflammation.

The procedure is described below.

### 1—Setting Up the Chronic Inflammation Model

After their arrival, the animals (Wistar rats, 280-300 g) were raised in 900 cm² cages (3 animals per cage). During this acclimatization period, the animals had free access to water and food. After this period, the animals (n=9) received, ad libitum, food and a solution of 2% (w/v) dextran sulfate sodium (DSS) in the drinking water. The induction of intestinal inflammation was monitored 3, 5 and 7 days after the start of the addition of DSS. A control group (n=3) received food and water ad libitum. If signs of pain were observed, the animal in question was isolated and paracetamol (200 mg/kg) was administered orally twice per day. These animals were removed from the experiment. If significant weight loss was observed, the animals were put down when they reached 80% of their initial weight. Lastly, animals were put down if they exhibited unusual behavior reflecting distress.

The conditions of accommodation were as follows: temperature 20-24° C., hygrometry 60 plus or minus 10%, 12 h/12 h cycle.

The animals were observed daily to watch for signs of stress or pain. Daily weighing of the animals, food and water was carried out. The appearance of clinical signs of inflammation was based on the loss of body weight, the consistency of stools (normal-0, soft-2, diarrhea-4) and rectal bleeding (0-4). The results made it possible to determine an inflammation severity index which represents the mean of the scores obtained for stool consistency and rectal bleeding. The weight loss represents the percentage difference in weight of the rats from D1 (addition of DSS) to D7.

### 2—Determining the “Effective” Dose

After their arrival, the animals (Wistar rats, 280-300 g) were raised in 900 cm² cages (5 animals per cage). During this acclimatization period, the animals had free access to water and food. After this period, the animals (n=30) were divided into 4 groups (Control−n=5, DSS+composition C1 10×−n=10). The “Control” group received food and water ad libitum throughout the experiment. The rats received, ad libitum, food and a solution of 2% DSS in the drinking water for 3, 5 or 7 days (duration determined during the first experiment). After the induction of intestinal inflammation, each rat received an intraperitoneal injection of 1 mL of composition C1. Composition C1 was administered once per day. If signs of pain were observed, the animal in question was isolated and paracetamol (200 mg/kg) was administered orally twice per day. These animals were removed from the experiment. If significant weight loss was observed, the animals were put down when they reached 80% of their initial weight. Lastly, animals were put down if they exhibited unusual behavior reflecting distress.

The conditions of accommodation were as follows: temperature 20-24° C., hygrometry 60 plus or minus 10%, 12 h/12 h cycle.

The animals were observed daily to watch for signs of stress or pain. Daily weighing of the animals, food and water was carried out.

The appearance of clinical signs of inflammation was based on the loss of body weight, the consistency of stools (normal-0, soft-2, diarrhea-4) and rectal bleeding (0-4).

The results made it possible to determine an inflammation severity index which represents the mean of the scores obtained for stool consistency and rectal bleeding. The weight loss represents the percentage difference in weight of the rats ((DX×D0)/D0)*100). At the end of the experiment, the animals were anesthetized (organ harvesting) and various samples were taken: blood, small intestine, cecum, colon, liver, spleen, and multiple parameters were evaluated: size of cecum, weight of liver, of spleen, and of cecum.

### Permeability Study

A permeability study was carried out on an isolated organ (jejunum or colon) in order to determine the efficacy of the dose of composition C1 on the recovery of intestinal permeability.

### Sampling Digestive Tract Fragments

This ex vivo approach used a fragment of jejunum or colon taken from the rat's digestive tract. The rats were anesthetized by inhaling isoflurane (1000 mg/g) at 3% for induction and 1.5% for maintenance during the operation. After laparotomy and ligation of the celiac artery against the esophagus, a 10 cm fragment of jejunum or colon was taken. The sample was weighed. The rats were then put down by injecting sodium pentobarbital (12 mg/100 g).

### Preparation of the Everted Organ and Sampling

The serosal and mucosal compartments of the organ were quickly rinsed with physiological saline solution (37° C.) and the organ was everted. A ligation was performed at one of the ends. The second end allows 1 mL of Krebs-Henseleit medium to be introduced. This end was then ligated, the organ was weighed and then placed in a container containing 10 mL of Krebs-Henseleit survival medium plus 250 mg of FITC-dextran, 4 kDa (mucosal compartment). Throughout the experiment, the everted organs were kept at 37° C. and oxygenated with an O2/CO2 mixture (95%/5%). Every 30 minutes, mucosal compartment medium was stirred by performing 3 suction-discharge cycles using a 1 mL micropipette. After 2 h of incubation, a sample of 1 mL was taken from the mucosal side.

The organs were removed from the glass container and one of the ends was cut. The serous medium was sampled and placed in a previously weighed tube. After sampling, the tube was again weighed in order to evaluate the transfer of survival medium over the course of the experiment. After weighing, the FITC-dextran was assayed by fluorimetry in duplicate (excitation, 490 nm; emission, 530 nm).

After weighing the organ, a longitudinal cut was made in the organ and it was quickly frozen in liquid nitrogen. The frozen organ was then placed in a zip bag, frozen in liquid nitrogen and then stored at −80° C.

### Study of Inflammation Parameters

The study was carried out based on the behavior of the animals treated. Specifically, inflammation causes exacerbated agitation in animals.

### Results

The results on the observation of the symptoms of inflammation are presented in tables 8 to 10 (before injection of composition C1) and in tables 11 to 15 (after injection of composition C1). In these tables:
the percent change in weight is calculated as follows: (DX×D0)/D0)*100)
Stool consistency is evaluated as follows: 0=normal; 2=soft; 3=diarrhea
Rectal bleeding is evaluated as follows: 0=none; 1=light; 2=moderate; 3=substantial; 4=profuse.

TABLE 8

| | | Days | | | | | |
|---|---|---|---|---|---|---|---|
| | | D3 | | | D4 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 10.7 | 0 | 0 | 12.14 | 0 | 0 |
| | Rat 2 | 11.1 | 0 | 0 | 13.02 | 0 | 0 |
| | Rat 3 | 9.5 | 0 | 0 | 16.07 | 0 | 0 |
| | Mean | 10.4 | 0 | 0 | 13.7 | 0 | 0 |
| DSS | Rat 4 | 5.2 | 0 | 0 | 8.52 | 0 | 0 |
| 2% | Rat 5 | 8.7 | 0 | 0 | 11.00 | 0 | 0 |
| | Rat 6 | 4.8 | 0 | 0 | 9.52 | 0 | 0 |
| | Mean | 6.2 | 0 | 0 | 9.7 | 0 | 0 |
| DSS | Rat 7 | 7.1 | 2 | 0 | 7.12 | 2 | 0 |
| 4% | Rat 8 | 3.6 | 2 | 0 | 5.57 | 2 | 0 |
| | Rat 9 | 6.2 | 2 | 0 | 6.49 | 2 | 0 |
| | Mean | 5.6 | 2 | 0 | 6.4 | 2 | 0 |

TABLE 9

| | | Days | | | | | |
|---|---|---|---|---|---|---|---|
| | | D5 | | | D6 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 11.07 | 0 | 0 | 14.04 | 0 | 0 |
| | Rat 2 | 13.65 | 0 | 0 | 16.51 | 0 | 0 |
| | Rat 3 | 14.75 | 0 | 0 | 17.74 | 0 | 0 |
| | Mean | 13.2 | 0 | 0 | 16.1 | 0 | 0 |
| DSS | Rat 4 | 8.52 | 0 | 0 | 10.03 | 0 | 0 |
| 2% | Rat 5 | 10.33 | 0 | 0 | 15.07 | 0 | 0 |
| | Rat 6 | 7.94 | 0 | 0 | 10.54 | 0 | 0 |
| | Mean | 8.9 | 0 | 0 | 11.9 | 0 | 0 |
| DSS | Rat 7 | 6.44 | 2 | 0 | 8.14 | 2 | 0 |
| 4% | Rat 8 | 8.20 | 2 | 0 | 9.64 | 2 | 0 |
| | Rat 9 | 5.84 | 2 | 0 | 7.40 | 2 | 0 |
| | Mean | 6.8 | 2 | 0 | 8.4 | 2 | 0 |

TABLE 10

| | | Days | | |
|---|---|---|---|---|
| | | D 7 | | |
| | | Weight | Stool | |
| | | change | consistency | Bleeding |
| | | (%) | (0/2/4) | (0-4) |
| Control | Rat 1 | 7.25 | 0 | 0 |
| | Rat 2 | 9.46 | 0 | 0 |
| | Rat 3 | 9.31 | 0 | 0 |
| | Mean | 8.67 | 0 | 0 |
| DSS 2% | Rat 4 | 4.59 | 2 | 0 |
| | Rat 5 | 6.50 | 2 | 0 |
| | Rat 6 | 4.06 | 2 | 0 |
| | Mean | 5.05 | 2 | 0 |
| DSS 4% | Rat 7 | 5.08 | 2 | 0 |
| | Rat 8 | 4.62 | 2 | 0 |
| | Rat 9 | 3.90 | 2 | 2 |
| | Mean | 4.53 | 2 | 1 |

TABLE 11

| | | Days | | | | | |
|---|---|---|---|---|---|---|---|
| | | D8 | | | D9 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 9.75 | 0 | 0 | 10.64 | 0 | 0 |
| | Mean | 6.53 | 2 | 0 | 3.13 | 2 | 0 |
| DSS | Rat 5 | 4.22 | 2 | 0 | 2.48 | 2 | 0 |
| 2% | Rat 6 | 5.38 | 2 | 0 | 2.80 | 2 | 0 |
| | Mean | 6.92 | 4 | 0 | 3.67 | 4 | 0 |
| DSS | Rat 8 | 1.36 | 4 | 2 | −1.10 | 4 | 4 |
| 4% | Rat 9 | 4.14 | 4 | 1 | 1.28 | 4 | 2 |
| | Mean | 9.75 | 0 | 0 | 10.64 | 0 | 0 |

TABLE 12

| | | Days | | | | | |
|---|---|---|---|---|---|---|---|
| | | D10 | | | D11 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 18.64 | 0 | 0 | 20.11 | 0 | 0 |
| | Mean | 3.17 | 2 | 0 | 5.87 | 2 | 0 |
| DSS | Rat 5 | 5.21 | 2 | 0 | 3.27 | 2 | 0 |
| 2% | Rat 6 | 4.19 | 2 | 0 | 4.57 | 2 | 0 |
| | Mean | 4.00 | 4 | 0 | 4.43 | 4 | 0 |
| DSS | Rat 8 | −0.88 | 4 | 4 | −0.36 | 4 | 0 |
| 4% | Rat 9 | 1.56 | 4 | 2 | 2.03 | 4 | 0 |
| | Mean | 18.64 | 0 | 0 | 20.11 | 0 | 0 |

TABLE 13

| | | Days | | | | | |
|---|---|---|---|---|---|---|---|
| | | D12 | | | D13 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 17.50 | 0 | 0 | 20.18 | 0 | 0 |
| | Mean | 3.73 | 2 | 0 | 3.63 | 2 | 0 |
| DSS | Rat 5 | 0.00 | 2 | 0 | −2.35 | 2 | 0 |
| 2% | Rat 6 | 1.87 | 2 | 0 | 0.64 | 2 | 0 |
| | Mean | 4.26 | 4 | 0 | 3.44 | 4 | 0 |
| DSS | Rat 8 | −1.30 | 4 | 0 | −2.18 | 4 | 0 |
| 4% | Rat 9 | 1.48 | 4 | 0 | 0.63 | 4 | 0 |
| | Mean | 17.50 | 0 | 0 | 20.18 | 0 | 0 |

TABLE 14

| | | Days | | | | | |
|---|---|---|---|---|---|---|---|
| | | D14 | | | D15 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 23.79 | 0 | 0 | 22.89 | 0 | 0 |
| | Mean | 6.53 | 0 | 0 | 2.87 | 0 | 0 |
| DSS | Rat 5 | 4.22 | 0 | 0 | −2.41 | 0 | 0 |
| 2% | Rat 6 | 5.38 | 0 | 0 | 0.23 | 0 | 0 |
| DSS | Mean | 6.92 | 4 | 0 | 2.66 | 4 | 2 |
| 4% | Rat 8 | 1.36 | 4 | 2 | −2.31 | 4 | 2 |
| | Rat 9 | 4.14 | 4 | 1 | 0.18 | 4 | 2 |
| | Mean | 23.79 | 0 | 0 | 22.89 | 0 | 0 |

TABLE 15

| | | Days | | | | | |
|---|---|---|---|---|---|---|---|
| | | D16 | | | D17 | | |
| | | | Stool | | | Stool | |
| | | Weight | consis- | Bleed- | Weight | consis- | Bleed- |
| | | change | tency | ing | change | tency | ing |
| | | (%) | (0/2/4) | (0-4) | (%) | (0/2/4) | (0-4) |
| Control | Rat 1 | 24.57 | 0 | 0 | 19.64 | 0 | 0 |
| | Mean | 3.20 | 0 | 0 | 0.93 | 0 | 0 |
| DSS | Rat 5 | −3.02 | 0 | 0 | −5.02 | 0 | 0 |
| 2% | Rat 6 | 0.09 | 0 | 0 | −2.04 | 0 | 0 |
| | Mean | 2.46 | 2 | 2 | 0.75 | 2 | 2 |
| DSS | Rat 8 | −3.31 | 2 | 2 | −2.95 | 2 | 2 |
| 4% | Rat 9 | −0.43 | 2 | 2 | −1.85 | 2 | 2 |
| | Mean | 24.57 | 0 | 0 | 19.64 | 0 | 0 |

It can be seen that the change in weight stabilized at D12 for the rats with DSS 2% and DSS 4%.

Regarding stool consistency, it becomes normal after 6 days of treatment with the composition according to the invention for the DSS 2% rats and after 8 days of treatment with the composition according to the invention for the DSS 4% rats.

Regarding rectal bleeding, it is absent in the DSS 2% rats, which means that there was recovery of intestinal function.

Regarding the analysis of the permeability of the intestinal membrane, the results of the in vivo tests 10 days after the injection of composition C1 into the DSS 2% rats, per FITC-dextran measurement, are presented in table 16.

TABLE 16

| | Control | DSS 2% | DSS 2% |
|---|---|---|---|
| | (FITC) - Rat 1 | FITC (Rat 5) | FITC (Rat 6) |
| Plasma | 0.6519 | 0.6024 | 0.7431 |

It can be seen that the assay of FITC-dextran, 4 kDa in plasma makes it possible to clearly discern the impact of composition C1 according to the invention on rats that received 2% DSS, the fluorescence found in the blood being identical to that of the control.

Regarding the analysis of the permeability of the intestinal membrane, the results of the ex vivo tests 10 days after the injection of composition C1 into the DSS 2% rats, on isolated organs (permeability for isolated organs was carried out on a fragment of distal jejunum (5 cm) and on the ileum (5 cm), per FITC-dextran measurement, are presented in table 17.

TABLE 17

| Organs isolated | 30 min | 60 min | 90 min | 120 min | 150 min |
|---|---|---|---|---|---|
| Control | 34.4 | 103.6 | 254.6 | 526.9 | 861.5 |
| (FITC) - Rat 1 | | | | | |
| DSS 2% | 30.7 | 137.9 | 457.2 | 841.4 | 1101.0 |
| FITC (Rat 5) | | | | | |
| DSS 2% | 27.4 | 85.3 | 293.0 | 659.6 | 1030.0 |
| FITC (Rat 6) | | | | | |

It can be seen that for the rats treated with composition C1 according to the invention that received 2% DSS, the values are close to those observed in the control.

### Molecular Analysis of Mouse Fecal Microbiota (Wild-Type—WT, SOD1, SOD1+Composition According to the Invention)

The SOD1 mouse is an animal model for axon degeneration consisting of transgenic mice expressing the mutated form of the human gene for superoxide dismutase. It is the in-vivo reference model for studying amyotrophic lateral sclerosis.

### Preparation of the Compositions:

The following amounts of conjugates were weighed in order to prepare a stock of 300 ml of composition C1. All of the conjugates were weighed using an analytical balance under a laminar flow hood.

TABLE 18

| PLL conjugate | Concentration | Concentration |
|---|---|---|
| (PLL = poly-L-lysine) | 1X (mg/mL) | 10X (mg/mL) |
| Oleyl-PLL | 0.063 | 0.63 |
| Palmitic-PLL | 0.024 | 0.24 |
| Lauryl-PLL | 0.059 | 0.59 |
| Linoleyl-PLL | 0.025 | 0.25 |
| Azelayl-PLL | 0.024 | 0.24 |
| Palmitoleyl-PLL | 0.049 | 0.49 |
| Thioctyl-PLL | 0.07 | 0.7 |
| Myristyl-PLL | 0.024 | 0.24 |
| Orotyl-PLL | 0.023 | 0.23 |
| Acetate-PLL | 0.055 | 0.55 |
| Buyrate-PLL | 0.089 | 0.89 |
| Lactate-PLL | 0.056 | 0.56 |
| Propionate-PLL | 0.055 | 0.55 |

Each conjugate was weighed in a sterile 50 ml centrifuge tube, and was dissolved in 30 ml of water, using a vortex mixer. Each conjugate was stirred for a period of between 10 and 20 minutes depending on the solubility of the conjugate.

In parallel, cholesterol and farnesyl cysteine molecules were dissolved separately at the approximate concentration of 50 mg/ml in absolute ethanol and filtered over a 0.22 μm filter. The solvent was evaporated using a rotary evaporator. The required amounts of dry solids were weighed under a laminar flow hood.

TABLE 19

| | Concentration | Concentration |
|---|---|---|
| Molecule | 1X (mg/mL) | 10X (mg/mL) |
| Cholesterol-PLL | 0.004 | 0.04 |
| farnesyl cysteine-PLL | 0.004 | 0.04 |

In a sterile 500 ml flask equipped with a magnetic bar, 23.8 ml of each conjugated solution prepared previously was added while being magnetically stirred (700 revolutions per minute). The corresponding amounts of (previously freeze-dried) salts at 300 of PBS were added to the solution of conjugates while being magnetically stirred (700 revolutions per minute).

Cholesterol and farnesyl cysteine were added and the mixture was stirred for 25 hours (700 revolutions per minute).

30 ml of formulation C1 10× was taken under a laminar flow hood and added to a new sterile 500 ml flask.

270 ml of sterile PBS was added to obtain formulation C1 1×. The formulation was stirred for 30 minutes.

The flasks were prepared according to the following plan using a sterile calibrated 5 ml pipette
C1 1×=83 flasks, 2 ml each.
C1 10×=83 flasks, 2 ml each.

The 166 flasks were placed in a steel freeze-drying unit and a freeze-drying cycle of one day was initiated.

Table 20 describes the composition of composition C2 used in this test.

TABLE 20

| PLL conjugate | Concentration | Concentration |
|---|---|---|
| (PLL = poly-L-lysine) | 1X (mg/mL) | 10X (mg/mL) |
| Alpha-tocopherol-PLL | 0.070 | 0.70 |
| Ascorbyl PLL | 0.063 | 0.63 |
| Biotinyl-PLL | 0.068 | 0.68 |
| GABA-PLL | 0.126 | 1.26 |
| Glutathione-PLL | 0.074 | 0.74 |
| Pantothenyl-PLL | 0.068 | 0.68 |
| Retinoyl-PLL | 0.202 | 2.02 |
| Spermine-PLL | 0.066 | 0.66 |
| Taurine-PLL | 0.110 | 1.10 |
| PLL/Cysteine (copolymer) | 0.06 | 0.6 |
| PLL/Methionine (copolymer) | 0.101 | 1.01 |

In this test, compositions C1 (described in table 18 and 19) and C2 (described in table 20) were injected one after the other with a one hour interval, starting with composition C1.

The feces of five female mice from each group (WT, SOD1, SOD1-PL-low dose and SOD1-PL-high dose), collected from the cages, were analyzed at T0 (=T6), T3 weeks (T9) and 10 weeks (T16), i.e. a total of 55 samples.

C1 and C2 together are referred to as “PL” for the rest of this test and the results.

In a first step, the total genomic DNA was extracted from each fecal sample using Godon's method (Godon et al, Appl Envion. Microbiol, 1997). The quality of the extracted DNA was evaluated after migration of the samples through agarose gel. DNA concentration was then determined using NanoDrop technology.

The DNA samples obtained were then sequenced. The approach used was high-throughput sequencing of the gene coding for 16S ribosomal RNA (V3-V4 region) using Illumina technology (read lengths: 150 bases, read depth: 1 million reads).

The taxonomic affiliation of each bacterial sequence (or OTU) obtained was carried out using the silva_nr99_v138.1 (https://www.arb-silva.de/) and GTDB v202 (https://gtdb.ecogenomic.org/) databases.

The rANOMALY pipeline (Theil S and Rifa E. rANOMALY: AmplicoN workflow for Microbial community AnaLYsis [version 1; peer review: 2 approved] FIOOOResearch 2021, 10:7 https://doi.org/10.12688/fl000research.27268.1), based on the DADA2 program, was used to process all of the sequences, whether to estimate the abundance of bacterial phyla/families or to carry out statistical analyses (composition, diversity analyses, differential abundance analyses).

The comparison of bacterial diversity and richness between the microbiota obtained from treated and untreated mice was carried out by calculating the Shannon index. The structural differences of the populations (β diversity) were revealed using two multivariate analysis methods (ANOVA and non-metric multidimensional scaling=MDS method) and a PLS-DA (partial least squares discriminant analysis) from the mixOmics packages.

The OTU differential abundance analyses were carried out using the normalization and estimation tools from the DESeq2 package [Love, M.I., Huber, W., Anders, S. Moderated estimation of fold change and dispersion for RNA-seq data with DESeq2 Genome Biology 15 (12): 550; 2014]

### Microbiota Correlation—Clinical Parameters

The potential correlation between each of the measured clinical and physiological parameters and the molecular composition of the microbiota was studied for each of the female mice for which the microbiota was analyzed (five mice per group).

Several analysis methods were tested, based on rank correlation (Spearman's rank correlation).

The method adopted is canonical correlation analysis (rCCA) provided by the mixOmics package (Rohart F, Gautier B, Singh A, and Le Cao K-A, 2017: mixOmics: An R package for ‘omics feature selection and multiple data integration. PLoS computational biology 13 (11): e1005752). This method makes it possible to perform cross-validation on the results of analyses of the correlations between the abundance of OTUs and the various clinical parameters measured. The results obtained are presented in the form of heat map showing significant, positive or negative, correlations between the abundance of OTUs and the clinical parameters.

### Results:

Comparison of the Composition of the Fecal Microbiota of “SOD1” KO Mice with “Wild-Type” (WT) Mice

The analysis of the relative abundance of phyla, orders, families or genera of bacteria present in the SOD1 mice relative to the WT mice shows that the composition of the microbiota of the SOD1s differs from that of the WTs:
the SOD1s have fewer Firmicutes and more Bacteroidetes than the WTs
Among Bacteroidetes, the SOD1 mice host more Bacteroidales, in particular Muribaculaceae and more Porphyromonadaceae, including Muribaculum, than the WTs
Among Firmicutes, the relative abundance of Lachnospiraceae (Shaedlerella and Acetifractor) and Erysipelotrichales is decreased whereas Lactobacillies such as Limolactobacillus and Oscillospiraceae, as well as Eggerthellaceae, show increased abundance relative to the WT mice

Analysis of the diversity (α diversity) and richness (β diversity) of the fecal microbiota of the SOD1 mice in comparison with that of the WT mouse made it possible to determine that there is:
no significant difference in bacterial diversity (index: Chao1, observed, Shannon and inverse Simpson indexes) between SOD1 and WT (a diversity).
a significant difference (Jaccard: p=0.01 and Bray-Curtis: p=0.01) in richness of bacterial species (β diversity). No time effect (T0, T6 and T16) was demonstrated.

The Jaccard and Bray-Curtis indexes or distances are two well-known metrics for measuring similarity, dissimilarity and diversity between multiple samples.

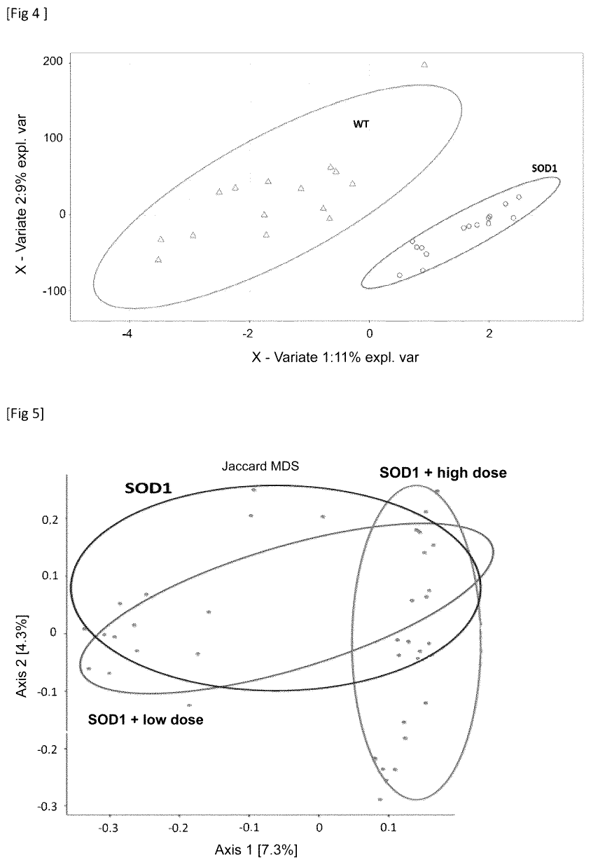

Lastly, the PLS-DA analysis showed that the fecal microbiota of the WT and SOD1 mice differ (FIG. 4).

In conclusion, SOD1 mice had disrupted intestinal microbiota compared with WT mice, characterized by less bacterial richness, a greater proportion of gram-negative bacteria (Muribaculaceae and Muribaculum) as well as gram-positive bacteria from Lactobacillies and Eggerthellaceae, but a lower proportion of Lachnospiraceae and Erysipelotrichales.

Effect of the Administration of PL at High or Low Dose in SOD1 mice

### A—Administration of a High Dose 10×

Administration of the composition at a high dose for three weeks leads to:
An increase in the relative abundance of:
Bacteroidales, in particular Muribaculaceae
Oscillospiraceae
COE1
A decrease in the abundance of:
Lachnospiraceae (Lactobacillus genus)
Lachnospiraceae

Administration of a high dose of the composition for three weeks appears to accentuate the disruptions observed in SOD1 mice.

### B—Administration of a Low Dose IX

Administration of a low dose of PL to SOD1 mice also caused changes in the relative abundance of certain microbial groups. Thus, the following were observed:
An increase in the relative abundance of:
Lachnospiraceae
Oscillospirales
GAC-485
A decrease in the relative abundance of:
Lactobacillies

The low dose seems to partly rebalance the intestinal microbiota of the SOD1 mice so as to approximate the composition of the microbiota of the wild-type (WT) mice, in particular by bringing about an increase in Lachnospiraceae and a decrease in Lactobacillis effect seems progressive since it is more pronounced after 10 weeks of treatment than after three weeks.

### Comparison of Bacterial Diversity and Richness in Treated and Untreated SOD1 Mice

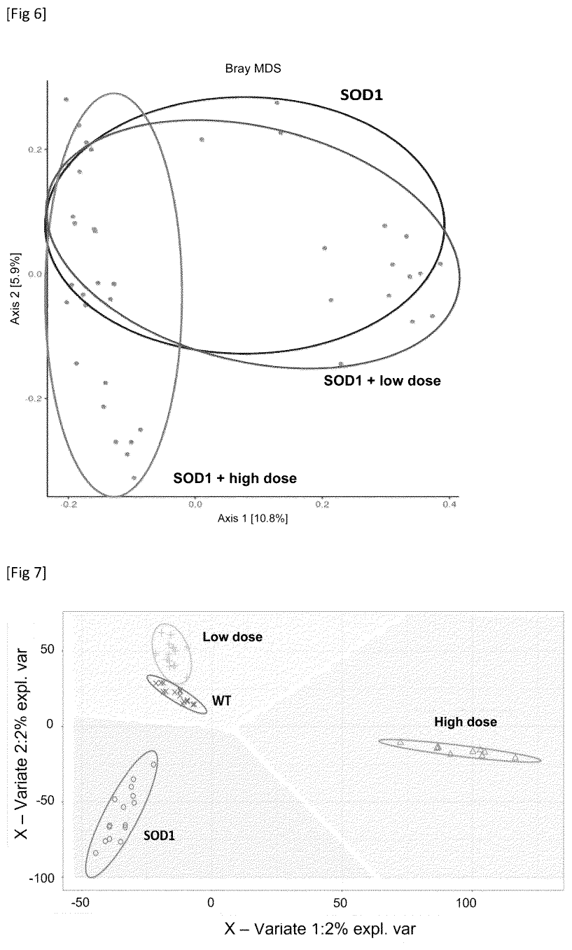

Analysis of the α diversity of the fecal microbiota did not demonstrate a significant effect from the administration of PL, at low and high doses, on bacterial diversity in SOD1 mice. A significant difference (Shannon index, p=0.02) was only observed between the fecal microbiota of mice treated with the low dose and those treated with the high dose of PL. (FIGS. 5 and 6).

Analysis of β diversity made it possible to show significant differences between untreated SOD1 mice and treated SOD1 mice.:
Controls versus PL high dose (p=0.02 Jaccard and p=0.01 Bray-Curtis)
PL low dose versus high dose (p=0.02 Jaccard and p=0.01 Bray-Curtis)
No significant difference between untreated control and PL low dose.
   Comparison Between Wild-Type Mice and SOD1 Mice Treated or Untreated with Different Doses of PL

PLS-DA of the composition of all of the microbiota studied made it possible to show that these microbiota could be separated into four distinct groups, with the microbiota of the mice treated with the low dose of PL coming close to—being in the same cluster as—the microbiota of the wild-type mice (FIG. 7).

### Conclusion

The study made it possible to show that the composition of the microbiota of the SOD1 mice differs from that of the wild-type mice, with these alterations affecting microbiota richness more than microbiota diversity. The microbiota of the SOD1 mice appears to be disrupted since the relative abundance of certain major bacterial groups is altered. It is characterized, in particular, by a higher abundance of Muribaculaceae and Lactobacillies (Oscillospiraceae) at the expense of Lachnospiraceae and Erysipelotrichales. The PLS-DA additionally demonstrated that the microbiota of wild-type and SOD1 mice were distinct.

The administration of a high dose of PL to the SOD1 mice for three weeks appears to amplify the differences observed between the wild-type mice and SOD1 mice (increase in Muribaculaceae and Oscillospiraceae and decrease in Lachnospiraceae). The composition of the microbiota of the SOD1 mice treated with the high dose of PL is very clearly different from those of the SOD1 controls as well as those of the wild-type mice, this group of mice treated with the high dose being substantially separated from the others (most disrupted microbiota).

The administration of a low dose of PL to the SOD1 mice for three to 10 weeks appears to partly rebalance the disrupted microbiota observed in the SOD1 control mice. Specifically, an increase in Lachnospiraceae and a decrease in Lactobacillies microbial groups is observed in the SOD1 mice relative to the wild-type mice. The composition of the microbiota of the SOD1 mice treated with a low dose of PL thus comes close to that of the wild-type mice, the microbiota of these two groups being grouped in the same cluster.

These preliminary results are of interest with regard to the administration of the lowest dose in the SOD1 model mouse. Specifically, at this dose, PL seems to have a beneficial effect on the disrupted microbiota of the SOD1 mice.

Study on Correlation Between Symptoms and Molecular Composition of the Microbiota in SOD1 Mice after Administration of a Low Dose of Polylysine (PL).

The objective of the study was to analyze potential correlations between the molecular composition of the intestinal microbiota and the clinical parameters measured in the SOD1 mice versus the wild-type mice and in the SOD1 mice treated or untreated with a low dose of PL for 10 weeks.

This study was carried out in female mice in which the molecular analysis of the fecal microbiota thereof was carried out beforehand (five females from each group [WT, SOD1, SOD1-low dose), collected from the cages, at TO (=T6), T3 weeks (T9) and 10 weeks (T16)].

Tests were carried out at least 1 hour after the first daily dose of the test or vehicle compound.

A session for one day comprised a 5 min practice run at 4 rpm on a rotary apparatus (AccuScan Instruments, Columbus, USA). One hour later, the animals were tested for three consecutive acceleration trials of 6 min with the speed going from 0 to 40 rpm over 360 s, with an inter-trial interval of at least 30 min. The latency to fall from the rod was recorded

### Study on Correlation in SOD1 and Wild-Type Mice.

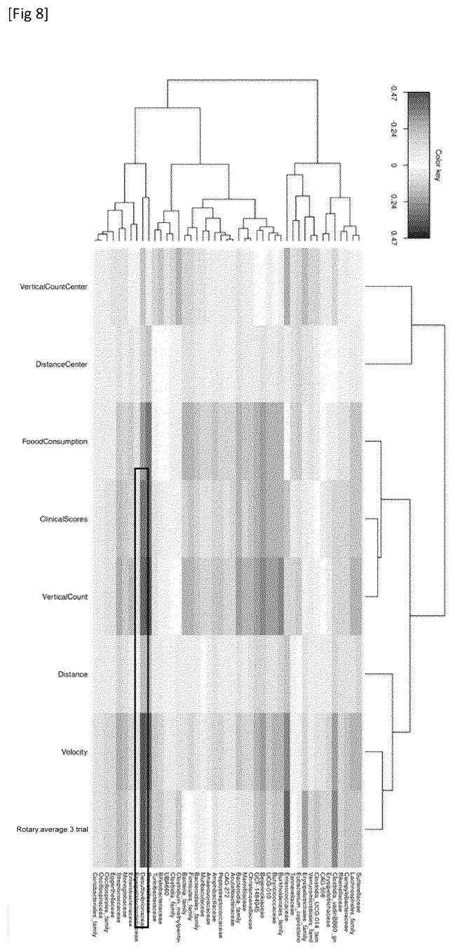

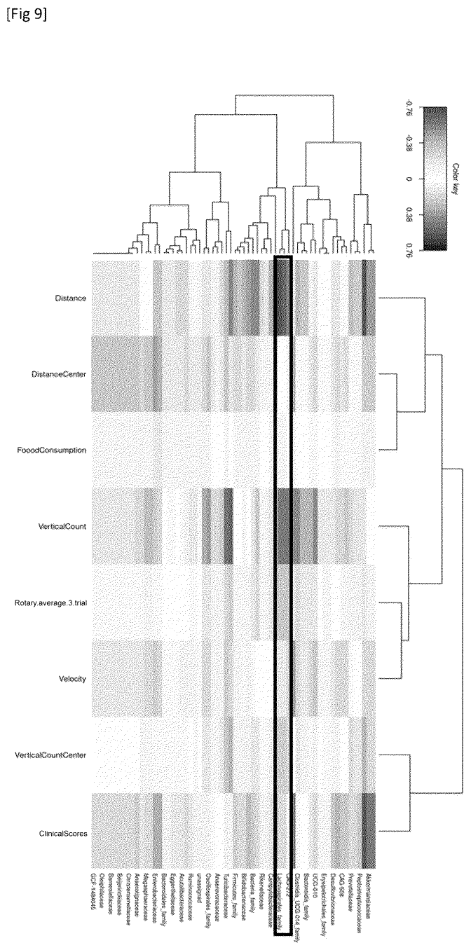

The RCCA (regularized canonical correlation analysis) mainly made it possible to show the presence of a significant negative correlation between clinical score, distance traveled and recovery time after Rotarod and the Desulfovibrionaceae family and a smaller negative correlation with the Prevotellaceae family in WT and SOD1 mice. Thus, the higher the abundance of these bacterial families, the lower the clinical score, and likewise for distance traveled and recovery time (FIG. 8).

Although the relative abundance of the Desulfovibrionaceae family (and of the Prevotellaceae family) is low compared to other bacterial families, the presence of the OTU for this family could only be found in the SOD1 mice (not detected in wild-type mice). This could suggest Desulfovibrionaceae playing a potential role in the physiopathology of SOD1 mice.

Study on Correlation in SOD1 Mice Treated or Untreated with the Low Dose of PL for 10 Weeks

The RCCA carried out in the SOD1 control mice and those treated with the low dose of PL made it possible to demonstrate a strong positive correlation between the Lachnospiraceae family and the distance traveled by the mice. The more abundant this family, the greater the distance traveled by the mice. Given that one of the main effects of PL on the intestinal microbiota of the SOD1 mice is to increase the abundance of Lachnospiraceae species, the observed correlation suggests this bacterial family playing a role in the recovery of the ability of the SOD1 mice to travel a distance close to that traveled by the wild-type mice (FIG. 6).

The demonstration of a negative correlation between Desulfovibrionaceae and the severity of symptoms in the SOD1 mice suggests that species from this family, which produce sulfides, may be involved in physiopathology, as has been observed in other pathologies such as irritable bowel syndrome or chronic inflammatory bowel diseases (Crohn's disease, hemorrhagic rectocolitis). In parallel, the lower abundance of species from the Lachnospiraceae family in the SOD1 mice could lead to a decrease in the concentration of intracolic butyrate, since a large proportion of the species from Lachnospiraceae produce this metabolite with demonstrated effects on health.

The administration of PL at a low dose for 10 weeks is accompanied by changes in the composition of the microbiota of the SOD1 mice, resulting, in particular, in an increase in the abundance of Lachnospiraceae and the disappearance of Desulfovibrionaceae. This correlates with a significant improvement in the ability of the mice to travel a distance equivalent to that of the wild-type mice. The administration of PL therefore partly rebalances the altered microbiota of the SOD1 mice, bringing about metabolic changes, and decreases the severity of certain symptoms in the SOD1 mice, or even restores them to a normal phenotype.

### Comparative Study on the Histopathological Analysis of SOD1, SOD1+Low Dose 1× and SOD1+High Dose 10× Mice.

SOD1 mice received the compound PL described in example 7 at low (1×) or high (10×) doses or a control vehicle (n=14/dose group) at the ages of 7 to 21 weeks.

In addition, 14 non-carrier wild-type (WT) mice received a control vehicle dose.

The animals were tested for their open-field behavior (at the ages of 6, 9, 13 and 15 weeks), their Rotarod performance (at the ages of six, nine, 13 and 15 weeks) and their grip strength (at the ages of 6, 9, 13, 15 and 17 weeks). An ophthalmoscopic analysis was carried out at the ages of 9, 13 and 17 weeks. Body weight, clinical symptoms, time to onset of disease and survival of the animals were monitored throughout the study. Blood, urine and feces were taken at the ages of 6, 9 and 16 weeks. Terminal samples were collected when an animal met a human endpoint criterion or reached the age of 150 days.

The histopathological analysis of 30 different tissue types was carried out.

| | | |
|---|---|---|
| SOD1 mice | SOD1 + 1X mice | SOD1 + 10X mice |

1. Spinal cord

| | | |
|---|---|---|
| Minimal to moderate | Minimal to pronounced | Minimal diffuse |
| diffuse vacuolization | diffuse vacuolization | degeneration |
| in males, slight to | in males, minimal | of spinal |
| pronounced diffuse | to moderate diffuse | nerve axons |
| vacuolization | vacuolization | in both sexes |
| in females. | in females | |

2. Brain

| | | |
|---|---|---|
| Multifocal vacuolization: | Slight or moderate | Minimal multifocal |
| slight or moderate in | vacuolization | vacuolization in |
| males, moderate in | in both sexes | females |
| females. | | |

3. Sciatic nerve

| | | |
|---|---|---|
| Slight or moderate | Slight or moderate | Minimal focal |
| degeneration of axons in | degeneration of | axonal |
| both sexes. | axons in both sexes. | degeneration |

4. Gastrocnemius muscle

| | | |
|---|---|---|
| Minimal to moderate | Slight or moderate | Minimal atrophy |
| atrophy in both sexes | atrophy in | in males and |
| | males, minimal to | in females |
| | moderate in females | |

5. Brown adipose tissue, aorta and/or kidneys

| | | |
|---|---|---|
| Slight to pronounced | Slight to pronounced | Slight diffuse |
| atrophy in both sexes | atrophy in both sexes | atrophy in males |
| | | and in females |

6. Spleen

| | |
|---|---|
| Necrosis of white pulp | Minimal or slight |
| lymphocytes: minimal in | necrosis of white |
| males, moderate in | pulp lymphocytes |
| females. | in females |

7. Thymus

| | |
|---|---|
| Moderate decrease in | Moderate decrease in |
| lymphocyte cellularity | lymphocyte cellularity |
| in males. Lymphocyte | in males and in females. |
| necrosis: slight or | Minimal to pronounced |
| moderate in males, | lymphocyte |
| moderate in females. | necrosis in females. |

The histopathological analysis shows that the administration of the compound according to the invention improves the condition of the tissues. More particularly, the spinal cord, the brain and the gastrocnemius muscle are tissues that exhibit deteriorated condition in the SOD1 mice. It can clearly be seen that the composition has an effect on these tissues depending on the dose used.

Thus, the composition shows a dose-dependent effect on the SOD1 mice, thereby demonstrating a promising therapeutic effect.

1. A composition comprising at least the following molecules:
oleic acid,
palmitic acid,
lauric acid,
linoleic acid,
azelaic acid,
farnesyl cysteine,
palmitoleic acid,
cholesterol,
thioctic acid,
myristic acid,
orotic acid,
acetic acid,
butyric acid,
lactic acid,
propionic acid,
and/or a salt and/or an ester and/or an anhydride of one or more of these molecules.

2. The composition of claim 1, characterized in that at least one of the molecules of the composition selected from among oleic acid, palmitic acid, lauric acid, linoleic acid, azelaic acid, palmitoleic acid, thioctic acid, myristic acid, orotic acid, acetic acid, butyric acid, lactic acid, propionic acid, salts of these acids, esters of these acids and anhydrides of these acids, is covalently conjugated to at least one molecule of a polymer selected from among polylysine, polyethylene glycol, polyornithine, polyarginine and polyhistidine.

3. The composition of claim 1, characterized in that it comprises micelles in which at least farnesyl cysteine and/or cholesterol and/or an ester of these molecules are encapsulated.

4. The composition of claim 3, characterized in that at least one of the micelles is formed by amphiphilic conjugates each consisting of at least one hydrophobic molecule covalently conjugated to a molecule of a polymer selected from among polylysine, polyethylene glycol, polyornithine, polyarginine and polyhistidine.

5. The composition of claim 4, characterized in that at least one micelle is formed by amphiphilic conjugates each consisting of at least one molecule selected from among oleic acid, palmitic acid, lauric acid, linoleic acid, palmitoleic acid, myristic acid, salts, esters and anhydrides of these fatty acids, covalently conjugated to a molecule of a polymer selected from among polylysine, polyethylene glycol, polyornithine, polyarginine and polyhistidine.

6. The composition of claim 2, characterized in that the one or more polymers are selected from among poly-L-lysine, polyethylene glycol, poly-L-ornithine, poly-L-arginine and poly-L-histidine.

7. The composition of claim 2, characterized in that the polylysine is a poly-L-lysine with a molecular weight of between 12000 and 20000 Da.

8. The composition of claim 2, characterized in that it comprises at least:
A. the following conjugates, each conjugate consisting of a molecule covalently bonded to a polylysine:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates,
one or more linoleyl-poly-L-lysine conjugates, and
B. farnesyl cysteine and cholesterol, and/or an ester of these molecules, encapsulated in micelles.

9. The composition of claim 8, characterized in that farnesyl cysteine and cholesterol, and/or an ester of these molecules, are encapsulated in micelles formed by one or more of the conjugates from list A.

10. The composition of claim 8, wherein the poly-L-lysine is replaced with another polylysine or with polyethylene glycol, a poly-L-ornithine, a poly-L-arginine or a poly-L-histidine.

11. The composition of claim 1, characterized in that it also comprises one or more pharmaceutically acceptable excipients.

12. The composition of claim 1, characterized in that it is in liquid form or in solid form.

13. A method for producing the composition of claim 1, characterized in that it comprises the following steps:
a. creating an amphiphilic premix: mixing, in an aqueous solution, one or more conjugates selected from among:
one or more oleyl-poly-L-lysine conjugates
one or more palmitic-poly-L-lysine conjugates
one or more lauryl-poly-L-lysine conjugates
one or more azelayl-poly-L-lysine conjugates
one or more palmitoleyl-poly-L-lysine conjugates
one or more thioctyl-poly-L-lysine conjugates
one or more myristyl-poly-L-lysine conjugates
one or more orotyl-poly-L-lysine conjugates
one or more acetate-poly-L-lysine conjugates
one or more buyrate-poly-L-lysine conjugates
one or more lactate-poly-L-lysine conjugates
one or more propionate-poly-L-lysine conjugates,
one or more linoleyl-poly-L-lysine conjugates, and
b. adding at least farnesyl cysteine and cholesterol to the amphiphilic premix and stirring so as to form micelles formed by one or more of the conjugates of the amphiphilic premix encapsulating the farnesyl cysteine and cholesterol.

14. The production method as claimed claim 13, characterized in that the stirring in step b. is carried out for at least 60 minutes at a stirring speed of 100 revolutions per minute or less.

15. The production method as claimed in claim 13, characterized in that it also comprises a step c. of separating the soluble and insoluble phases, in order to recover the soluble phase.

16. The composition as claimed in claim 1, for use thereof as a drug.

17. The composition as claimed in claim 1, for use thereof in the prevention and/or treatment of a disease selected from among inflammatory diseases, neurodegenerative diseases, bacterial diseases, viral diseases, autoimmune diseases, allergies and food intolerances.

18. The composition as claimed in claim 1, for use thereof in the prevention and/or treatment of a disease selected from among eczema, psoriasis, chronic inflammatory bowel diseases, Alzheimer's disease, Parkinson's disease, multiple sclerosis, and amyotrophic lateral sclerosis.

19. The composition for use thereof as claimed in claim 16, associated with the use of a second composition comprising the following molecules:
taurine,
cysteine,
methionine
spermine
retinoic acid,
pantothenic acid,
biotin,
coenzyme Q10,
glutathione,
ascorbic acid,
GABA,
alpha-tocopherol
and/or a salt and/or an ester and/or an anhydride of one or more of these molecules.

20. The composition for use thereof as claimed in claim 19, characterized in that, in the second composition, at least one of the molecules of the composition selected from among taurine, spermine, pantothenic acid, retinoic acid, biotin, glutathione, ascorbic acid, GABA, and alpha-tocopherol, salts of these molecules, esters of these molecules, and anhydrides of these molecules is covalently conjugated to a molecule of a polymer selected from among polylysine, polyethylene glycol, polyornithine, polyarginine and polyhistidine.

21. The composition for use thereof as claimed in claim 19, characterized in that, in the second composition, cysteine and methionine are each conjugated to a molecule of a polymer selected from among polylysine, polyethylene glycol, polyornithine, polyarginine and polyhistidine by polymerization, said molecule and said polymer forming a copolymer.

22. The composition for use thereof as claimed in claim 19, characterized in that, in the second composition, coenzyme Q10 is encapsulated in micelles.

23. The composition for use thereof as claimed in claim 22, characterized in that at least one micelle of the second composition is formed by amphiphilic conjugates each consisting of at least molecule covalently conjugated to a molecule of a polymer selected from among polylysine, polyethylene glycol, polyornithine, polyarginine and polyhistidine.

24. The composition for use thereof as claimed in claim 22, characterized in that at least one micelle of the second composition is formed by amphiphilic conjugates each consisting of at least one molecule selected from among taurine, spermine, pantothenic acid, biotin, glutathione, ascorbic acid, GABA, and alpha-tocopherol, salts of these molecules, esters of these molecules, and anhydrides of these molecules covalently conjugated to a molecule of a polymer selected from among polylysine, polyethylene glycol, polyornithine, polyarginine and polyhistidine.

25. The composition for use thereof as claimed in claim 19, characterized in that the one or more polymers of the second composition are selected from among poly-L-lysine, polyethylene glycol, poly-L-ornithine, poly-L-arginine and poly-L-histidine.

26. The composition for use thereof as claimed in claim 19, in the prevention and/or treatment of pathological dysbiosis of the intestinal microbiota.

27. The composition for use thereof as claimed in claim 19, in the prevention and/or treatment of an intestinal disease associated with pathological dysbiosis of the intestinal microbiota.

28. The composition for use thereof as claimed in claim 19, in the prevention and/or treatment of an intestinal disease selected from among Crohn's disease, chronic inflammatory bowel diseases, hemorrhagic rectocolitis, irritable bowel syndrome, ulcerative colitis, rheumatoid arthritis and gluten intolerance.
